(19) United States
(12) Patent Application Publication
(10) Pub. No.: US 2024/0260584 A1
(43) Pub. Date: Aug. 8, 2024

(54) BACTERIAL BIO-CONTROL AGENTS FOR IMPROVING PLANTS' GROWTH, YIELD AND RESISTANCE TO PATHOGENS

(71) Applicant: THE STATE OF ISRAEL, MINISTRY OF AGRICULTURE & RURAL DEVELOPMENT, AGRICULTURAL RESEARCH ORGANIZATION, Rishon Lezion (IL)

(72) Inventor: Maya BAR, Ramat Gan (IL)

(21) Appl. No.: 18/276,585

(22) PCT Filed: Feb. 10, 2022
(86) PCT No.: PCT/IL2022/050165

§ 371 Date: Aug. 9, 2023

Related U.S. Application Data
(60) Provisional application No. 63/147,770, filed on Feb. 10, 2021.

(51) Int. Cl. A01N 63/20; A01N 63/22; A01N 63/27; A01P 1/00; A01P 3/00; A01P 21/00

(52) U.S. Cl. CPC A01N 63/20; A01N 63/22; A01N 63/27; A01P 1/00; A01P 3/00; A01P 21/00

(57) ABSTRACT

A method for improving a plant trait, comprising steps of: a. obtaining a plant; and b. inoculating said plant with at least one bacterium having at least one of sequences SEQ ID NO:1-SEQ ID NO: 17, as disclosed in the sequence listing of said application, germinating the seed and growing the plant A bacterial formulation useful for the method is disclosed comprising at least one bacterium having at least one of sequences SEQ ID NO:1-SEQ ID NO: 17, as disclosed in the sequence listing of said application.

### SEQUENCE LISTING

The instant application contains a Sequence Listing which has been submitted electronically in ASCII txt. format and is hereby incorporated by reference in its entirety. Said ASCII copy, is named seq.listing 1371-T-33-US and is 30 KB in size.

### FIELD OF THE INVENTION

The present disclosure relates to the fields of agrotechnology and phytopathology. More particularly, the present disclosure concerns the beneficial use of bacterial strains isolated from tomato plants in improving plants' growth, activating their immune response and priming the plants against pathogens.

### BACKGROUND OF THE INVENTION

The management of plant diseases is crucial for the production of quality and abundance of food, feed and fiber for maintaining the global food security demand that is estimated to increase by at least 70% until 2030. To cope with this increasing food demand, and given the limited availability of additional agricultural areas, the development of ecofriendly and effective strategies for management of plant diseases is highly necessary. Most of the plant diseases management strategies currently employed depend on cultural practices, such as the use of disease-resistant cultivars and crop rotation, which are predominantly preventive, as well as on synthetic chemical-based pesticides and fertilizers (see Raymaekers et al., 2020: Savary and Willocquet, 2020). Despite their success in plant diseases management, concerns about the harmful impact of chemical pesticides on human health have emerged, including environmental hazards and the development of resistant pests and pathogens (see Rahman et al., 2018: Marian and Shimizu, 2019). As an outcome of these problems, stringent laws and regulations have been enforced for the development of ecofriendly management approaches. Thus, agricultural scientists and farmers have diverted their attention towards the exploitation of approaches based on bio-pesticides.

A substitute to chemical/synthetic treatments is the exploitation of plant-associated beneficial microbes, as biological control agents (BCAs), since they can promote crop production and resistance to various plant diseases (see Köhl et al., 2019; Raymaekers et al., 2020). Previous research works have repeatedly demonstrated that several plant diseases can be managed by natural potential BCAs (see Rahman et al., 2018; Raymaekers et al., 2020). BCAs can mitigate the growth of phytopathogens by various modalities. Within the bacterial BCAs, the genera Bacillus and Pseudomonas are the most studied (see Shafi et al., 2017; Sun et al., 2017), due to their biocontrol and plant growth promotion properties. Bacillus species have become attractive biological control agents due to their ability to replicate rapidly, produce resistant endospores, and exhibit a broad spectrum of biocontrol abilities against a wide range of plant pathogens. BCAs can potentially produce antimicrobial chemicals, cause competition for space and nutrients, efficiently colonize plant roots, and activate host defensive mechanisms (see Santoyo et al., 2012; Ciancio et al., 2016). During the interaction of beneficial microbes with the plant roots, effective bacterial root colonizers can exert local antagonism or stimulate the plant systemic resistance, leading to rapid defense responses towards subsequent pathogen attacks (see Macho and Zipfel, 2014: Wang et al., 2020).

The plant immune response is regulated by several defense phytohormones i.e. salicylic acid (SA), jasmonic acid (JA) and ethylene (ET). These phytohormones control various aspects of the plant's life, such as seed production and reproduction, flowering, photosynthesis and response to environmental challenges. BCAs implement several sophisticated molecular mechanisms, such as systemic acquired resistance (SAR) and induced systemic resistance (ISR) to activate plant defense against various pathogens and pests (see Choudhary and Johri, 2009). SAR can be elicited by exposing the plant to virulent, avirulent, and non-pathogenic microbes. Depending on the plant species and microbial elicitors, a set period of time is required for the establishment of SAR wherein pathogenesis-related proteins (PRs: chitinase and glucanase) and accumulation of salicylic acid (SA) takes place. Contrasting SAR, ISR is not accompanied by PR-protein and SA accumulation: but instead, depends on the pathways regulated by jasmonate and ET. With regards to bacterial BCAs, earlier reports on various plants mention that different bacterial strains could have diverse effects in defense signaling induction. Hence, it is essential to study the expression of genes related to both signaling pathways, SA and JA/ET, to conclude the systemic resistance triggering effects of a single BCA. In light of the above, there is still an unmet need for developing means and methods which harness beneficial bacterial microorganisms as control agents for promoting growth, defense responses and induced resistance in plants.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention.

FIG. 1 depicting a schematic presentation of a phylogenetic tree showing the bacterial biological control agents:

FIG. 2A and FIG. 2B depicting a graphical presentation of the effect of the bacterial biological control agents of the present invention on plants' pathogens ex planta:

FIG. 3A and FIG. 3B depicting a graphical presentation of the effect of the bacterial biological control agents of the present invention on plants' height:

FIG. 4A, FIG. 4B and FIG. 4C depicting a graphical presentation of the effect of the bacterial biological control agents of the present invention on various plants' traits:

FIG. 5 depicting a graphical presentation of the effect of the bacterial biological control agents of the present invention on fungal disease development in planta:

FIG. 6A and FIG. 6B depicting a graphical presentation of the effect of the bacterial biological control agents of the present invention on plants' pathogens in planta:

FIG. 7A and FIG. 7B depicting a graphical presentation of the effect of the bacterial biological control agents of the present invention on ethylene production:

FIG. 8 depicting a graphical presentation of the effect of the bacterial biological control agents of the present invention on reactive oxygen species production:

FIGS. 9A-9D depicting a graphical presentation of a relative defense-related gene expression in tomato plants treated with the biological control agents of the present application; and

FIG. 10A-10D depicting a graphical presentation of a relative defense-related gene expression in tomato plants treated with the bacterial biological control agents of the present application.

FIG. 11. Depicting a Comparison of bacterial isolate activity across pathogen taxa

FIG. 12. Depicting a Comparison of bacterial isolate activity across plant developmental ages.

FIG. 13. Depicting Comparison of bacterial isolate activity across plant developmental ages.

FIG. 14. Depicting the Effect of seed coating with bacterial isolates on plant development.

### SUMMARY OF THE INVENTION

It is one object of the present invention to disclose a method for improving a plant trait, comprising steps of:
a. obtaining a plant; and
b. inoculating said plant with at least one bacterium having at least one of sequences SEQ ID NO:1-SEQ ID NO:17, as disclosed in the sequence listing of said application.

It is another object of the present invention to disclose the method as described above, wherein said plant is a tomato plant.

It is another object of the present invention to disclose the method as described above, wherein said plant is a seedling.

It is another object of the present invention to disclose the method as described above, wherein said plant trait is selected from a group consisting of improved height, increased number of flowers, increased number of fruits, improved weight, improved harvest index, activation of the immune response, enhanced resistance to pathogenic infections and any combination thereof.

It is another object of the present invention to disclose the method as described above, wherein said plant trait is characterized by an increase of at least about 2% compared to uninoculated plant of the same species.

It is another object of the present invention to disclose the method as described above, wherein said activation of the immune response is measured by a mean selected from a group consisting of production of ethylene, generation of reactive oxygen species, expression of genes related to the plant immune and defense response and any combination thereof.

It is another object of the present invention to disclose the method as described above, wherein said enhanced resistance to pathogenic infections is characterized by a decrease in a lesion area or the number of pathogenic microorganisms of at least about 2%.

It is another object of the present invention to disclose the method as described above, wherein said pathogenic infections are caused by microorganisms, selected from the group consisting of bacteria, fungi, viruses and any combination thereof.

It is another object of the present invention to disclose the method as described above, wherein said pathogenic bacteria is Xanthomonas campestris pv. vesicatoria.

It is another object of the present invention to disclose the method as described above, wherein said pathogenic fungi is Botrytis cinerea.

It is another object of the present invention to disclose the method as described above, wherein said bacterium is selected from a group consisting of Bacillus spp, Enterobacter spp, Massilia spp, Pseudomonas spp, Ralstonia spp and any combination thereof.

It is another object of the present invention to disclose the method as described above, wherein said Bacillus spp are selected from a group consisting of Bacillus cereus. Bacillus licheninformis, Bacillus subtilis, Bacillus megaterium, Bacillus aryabhattai, Bacillus pumulis and any combination thereof.

It is another object of the present invention to disclose the method as described above, wherein said Enterobacter spp is Enterobacter asburiae.

It is another object of the present invention to disclose the method as described above, wherein said Ralstonia spp is Ralstonia pickettii.

It is another object of the present invention to disclose the method as described above, wherein said Pseudomonas spp is Pseudomonas aeruginosa.

It is also an object of the present invention to disclose a bacterial formulation comprising at least one bacterium having at least one of sequences SEQ ID NO:1-SEQ ID NO:17, as disclosed in the sequence listing of said application.

It is another object of the present invention to disclose the bacterial formulation as described above, wherein said formulation comprises bacteria selected from a group consisting of Bacillus spp, Enterobacter spp, Massilia spp, Pseudomonas spp, Ralstonia spp and any combination thereof.

It is another object of the present invention to disclose the bacterial formulation as described above, for use in improving a plant trait.

It is another object of the present invention to disclose the bacterial formulation as described above, wherein said plant is a tomato plant.

It is another object of the present invention to disclose the bacterial formulation as described above, wherein said plant is a seedling.

It is another object of the present invention to disclose the bacterial formulation as described above, wherein said plant trait is characterized by an increase of at least about 2% compared to plant of the same species not treated with said bacterial formulation.

It is another object of the present invention to disclose the bacterial formulation as described above, wherein said plant trait is selected from a group consisting of improved height, increased number of flowers, increases number of fruits, improved plant weight, increased harvest index, activation of the immune response, enhanced resistance to pathogenic infections and any combination thereof.

It is another object of the present invention to disclose the bacterial formulation as described above, wherein said enhanced resistance to pathogenic infections is characterized by a decrease in a lesion area or the number of pathogenic microorganisms of at least about 2%.

It is another object of the present invention to disclose the bacterial formulation as described above, wherein said pathogenic infections are caused by microorganisms, selected from the group consisting of bacteria, fungi, viruses and any combination thereof.

It is another object of the present invention to disclose the above mentioned method for improving a plant trait wherein said plant has not yet set fruit.

It is one object of the present invention to disclose the method for improving a plant trait wherein said plant has set fruit.

It is another object of the present invention to disclose the above mentioned method for improving a plant trait wherein said plant is 3-6 weeks old.

It is another object of the present invention to disclose the above mentioned method for improving a plant trait wherein said plant is selected from a group consisting of a cucumber plant, a pepper plant, an eggplant plant, a potato plant, and a grapevine plant.

It is one object of the present invention to disclose the method for improving a plant trait comprising steps of:
a. obtaining a seed;
b. inoculating said seed with at least one bacterium having at least one of sequences SEQ ID NO:1-SEQ ID NO:17, as disclosed in the sequence listing of said application; and
c. germinating said seed and growing the plant.

It is another object of the present invention to disclose the above mentioned method for improving a plant trait wherein said plant is a tomato plant.

It is another object of the present invention to disclose the above mentioned method for improving a plant trait wherein said plant trait is selected from a group consisting of improved height, increased number of flowers, increased number of fruits, increased yield, improved weight, improved harvest index, activation of the immune response, enhanced resistance to pathogenic infections and any combination there

It is another object of the present invention to disclose the above mentioned method for improving a plant trait wherein said plant trait is characterized by an increase of at least about 2% compared to uninoculated plant of the same species.

It is one object of the present invention to disclose the the above mentioned method for improving a plant trait wherein said activation of the immune response is measured by a mean selected from a group consisting of production of ethylene, generation of reactive oxygen species, expression of genes related to the plant immune and defense response and any combination thereof

It is another object of the present invention to disclose the above mentioned method for improving a plant trait wherein said enhanced resistance to pathogenic infections is characterized by a decrease in a lesion area, decrease in tissue damage or the number of pathogenic microorganisms of at least about 2%.

It is another object of the present invention to disclose the above mentioned method for improving a plant trait wherein said pathogenic infections are caused by microorganisms, selected from the group consisting of bacteria, fungi, viruses and any combination thereof.

It is one object of the present invention to disclose the above mentioned method for improving a plant trait wherein said bacteria is Xanthomonas campestris pv. Vesicatory

It is one object of the present invention to disclose the above mentioned method for improving a plant trait wherein said fungi is Botrytis cinerea.

It is one object of the present invention to disclose the above mentioned method for improving a plant trait wherein said bacterium is selected from a group consisting of Bacillus spp, Enterobacter spp, Massilia spp, Pseudomonas spp, Ralstonia spp and any combination thereof.

It is one object of the present invention to disclose the above mentioned method for improving a plant trait wherein said Bacillus spp are selected from a group consisting of Bacillus cereus. Bacillus licheninformis, Bacillus subtilis, Bacillus megaterium, Bacillus aryabhattai, Bacillus pumulis and any combination thereof.

It is another object of the present invention to disclose the above mentioned method for improving a plant trait wherein said Enterobacter spp is Enterobacter asburiae.

It is one object of the present invention to disclose the above mentioned method for improving a plant trait wherein said Ralstonia spp is Ralstonia pickettii.

It is one object of the present invention to disclose the above mentioned method for improving a plant trait wherein said Pseudomonas spp is Pseudomonas aeruginosa.

It is an object of the present invention to disclose a bacterial formulation for improving a plant trait comprising at least one bacterium having at least one of sequences SEQ ID NO: 1-SEQ ID NO:17, as disclosed in the sequence listing of said application.

It is one object of the present invention to disclose the above mentioned formulation comprising bacteria selected from a group consisting of Bacillus spp, Enterobacter spp, Massilia spp, Pseudomonas spp, Ralstonia spp and any combination thereof.

It is one object of the present invention to disclose the bacterial formulation for use in improving a plant trait.

It is one object of the present invention to disclose the above mentioned bacterial formulation wherein said plant is a tomato plant.

It is one object of the present invention to disclose the above mentioned method for improving a plant trait

It is one object of the present invention to disclose the above mentioned bacterial formulation wherein said plant is a seedling.

It is one object of the present invention to disclose the above mentioned bacterial formulation wherein said plant trait is characterized by an increase of at least about 2% compared to plant of the same species not treated with said bacterial formulation.

It is one object of the present invention to disclose the method for improving a plant trait by using the above mentioned bacterial formulation, wherein said plant trait is selected from a group consisting of improved height, increased number of flowers, increases number of fruits, improved plant weight, increased harvest index, activation of the immune response, increased yield, enhanced resistance to pathogenic infections and any combination thereof.

It is one object of the present invention to disclose the method for improving a plant trait by using the above mentioned bacterial formulation, wherein said enhanced resistance to pathogenic infections is characterized by a decrease in a lesion area, decrease in tissue damage, or the number of pathogenic microorganisms of at least about 2%

It is one object of the present invention to disclose the above mentioned method for improving a plant trait by using the above mentioned bacterial formulation, wherein said pathogenic infections are caused by microorganisms, selected from the group consisting of bacteria, fungi, viruses and any combination thereof.

### DETAILED DESCRIPTION OF THE PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a method for

As used herein after, the term “about” refers to any value being up to 25% lower or greater the defined measure.

As used herein after, the term “biological control agents (BCAs)/bio-agents/biocontrol agents/bacterial biocontrol agents/bacterial bio-agents” refers to microorganisms which naturally colonize plants, but cause their hosts no harm. Said microorganisms can be considered beneficial, as they occupy space, otherwise inhabited by pathogens or harmful microorganisms, and they are also known for secreting compounds/chemicals which trigger the plant's defense mechanisms, thus, priming the plants for future pathogens' attacks. In the present application, BCAs are also named “bacterial isolates”, as they refer to strains of bacteria which naturally inhabit tomato plants. These strains were isolated from the plants' leaves, identified and used in the experiments described in the specification of the present application to evaluate their beneficial effects on plants' growth, yield-related traits, immune response and resistance to harmful pathogens.

As used herein after, the term “harvest index” refers to the ratio between the total fruit yield mass and total biomass.

The present application provides a method for inducing growth and defense and immune mechanisms in plants using bacteria, more particularly beneficial bacterial strains isolated from tomato plants. The use of said beneficial bacteria has significant agrotechnological importance, as it can be employed to improve plants' performance and traits, such as height, number of flowers/fruits, weight etc., and to enhance the plant immune response and prime it to better cope with pathogenic microorganisms.

The present application also provides a formulation, which comprises at least one bacterial strain having at least one of sequences SEQ ID NO: 1-SEQ ID NO:17 as disclosed in the present invention. In a preferred embodiment of the present invention, plants are inoculated with said formulation 1-3 days prior to transplanting in fields, greenhouses, research settings etc. and exhibit, as they grow, improved yield-related traits and enhanced immune and defense responses against pathogenic microorganisms, such as fungi and bacteria. In other non-limiting ways, plants at various developmental stages (seeds, germinated seeds, seedlings, mature plants, senesced plants) can be inoculated with the bacterial formulation of the present invention at various time points.

In a preferred embodiment of the present invention, the BCAs are isolated from tomato mutants or genotypes which are more resistant to plant diseases compared to wild type plants.

In yet another preferred embodiment of the present invention, the BCAs are bacterial strains, isolated from tomato leaves.

In yet another preferred embodiment of the present invention, the BCAs comprise at least one species of the Bacillus genus.

In yet another preferred embodiment of the present invention, plants inoculated with at least one of the BCAs of the present invention exhibit improved traits, such as improved height, increased number of inflorescences, improved weight, improved yield and biomass, as compared to tomato plants which are not treated with said BCAs.

In yet another preferred embodiment of the present invention, plants inoculated with at least one of the BCAs of the present invention produce more ethylene and reactive oxygen species (ROS) compared to untreated plants. Ethylene and ROS production are known markers for activation of the plant immune response.

In yet another preferred embodiment of the present invention, plants inoculated with at least one of the BCAs of the present invention, express genes which are tightly related with the plant immune responses.

In yet another preferred embodiment of the present invention, in plants inoculated with at least one of the BCAs of the present invention, the development and severity of a disease caused by plant pathogens are reduced in comparison with untreated plants.

In yet another preferred embodiment of the present invention, the BCAs of the present invention have no direct antagonistic activity against plants' bacterial and fungal pathogens, but those BCAs do activate the plant immune response and reduce pathogen infection in planta.

All experimental data disclosed in the following examples are presented as average±SEM. Differences between two groups were analyzed for statistical significance using a two-tailed t-test. Differences among three groups or more were analyzed for statistical significance with a one-way ANOVA. Regular ANOVA was used for groups with equal variances, and Welch's ANOVA for groups with unequal variances. When a significant result for a group in an ANOVA was returned, significance in differences between the means of different samples in the group were assessed using a post-hoc test. The Tukey test was employed for samples with equal variances when the mean of each sample was compared to the mean of every other sample. The Bonferroni test was employed for samples with equal variances when the mean of each sample was compared to the mean of a control sample. The Dunnett test was employed for samples with unequal variances. All statistical analyses were conducted using Prism⁸™.

### Example 1

To identify bacterial strains inhabiting tomato plants with the potential of functioning as biological control agents, tomato leaf samples were collected from ARO, The Volcani center, Rishon lesion, Israel, during the summer of 2018. For epiphytic bacteria isolation, tomato leaves (1 g) were placed in 5 ml sterilized distilled water and kept on a 100-rpm shaker for 30 minutes. A 50) ul of the resulting suspension was spread onto different bacterial media such as LB (Luria-Bertani) medium, nutrient agar medium and YPGA (Yeast extract: 7 g/l, Peptone: 7 g/l, Glucose: 7 g/l, Agar: 15 g/l). The plates were incubated at 28° C. for 24-48 h. All bacterial isolates were purified on LB medium. Further, the bacterial strains were suspended in 30% glycerol in cryogenic tubes and kept at −80° C. for the long-term storage. The purified bacterial isolates genomic DNA was amplified using bacterial primers 27f (5′-AGAGTTTGATCTGGCTCAG-3′) and 1492r (5′-GGTTACCTTGTTACGACTT-3′). PCR was performed by means of a Thermal cycler: 94° C. for 3 min (1 cycle): 94° C. for 1 min, 55° C. for 45 s and 72° C. for 1.5 min (15 cycles); and 72° C. for 10 min (1 cycle). PCR products (approx. 1500 bp) were purified by PCR purification kit (Hylabs) following the protocol of the manufacturer and were sent to an external company for sequencing (Hylabs, Israel). The obtained chromatograms were visually inspected and sequences obtained were compared with those from the EZTaxon database: aligned using the Clustal W software and phylogenetic trees inferred using the neighbor-joining method in the MEGA X program (see Chun et al., 2007).

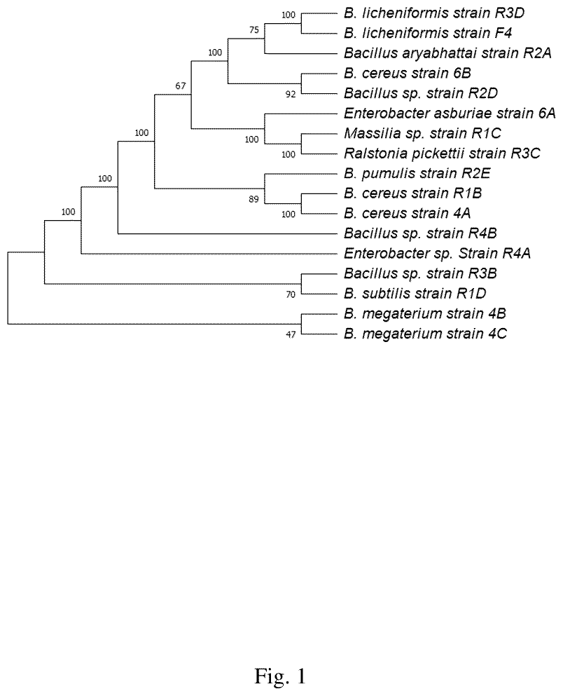

The identification of these bacteria using 16S rRNA gene sequencing revealed that most of the bacteria collected from tomato leaves belonged to the genus Bacillus spp. Reference is now made to FIG. 1 depicting phylogenetic relationships between the bacterial isolates to known species in NCBI GenBank nucleotide sequence database. The isolates R3B, R4B and R2D showed 95%, 86% and 100% similarity with Bacillus sp. respectively, isolates 6B, RIB and 4A with B. cereus (97%, 100% and 97%, respectively), isolates R3D, and F4 with B. licheniformis (100% and 99%, respectively), isolate RID with B. subtilis (95%), isolate R2E with B. pumulis (97%), isolates 4B and 4C with B. megaterium (96%), isolate R2A with B. aryabhatti (95%), isolates R4A and 6A with Enterobacter (94%) and E. asburiae (100%), respectively, RIC with Massilia sp. (95%) and R3C with Ralstonia pickettii (97%).

The 16S rRNA gene sequences of the above-mentioned bacteria are detailed in the sequence listing provided with the present application. The sequences are as follows:
1) 16S rRNA gene of Bacillus sp. strain R3B referred in the present disclosure as SEQ ID NO:1;
2) 16S rRNA gene of Bacillus sp. strain R4B referred in the present disclosure as SEQ ID NO:2;
3) 16S rRNA gene of B. cereus strain 6B referred in the present disclosure as SEQ ID NO:3;
4) 16S rRNA gene of B. cereus strain RIB referred in the present disclosure as SEQ ID NO:4;
5) 16S rRNA gene of B. licheniformis strain R3D referred in the present disclosure as SEQ ID NO:5;
6) 16S rRNA gene of B. licheniformis strain F4_27 F referred in the present disclosure as SEQ ID NO:6;
7) 16S rRNA gene of B. subtilis strain RID referred in the present disclosure as SEQ ID NO:7;
8) 16S IRNA gene of B. cereus strain 4A referred in the present disclosure as SEQ ID NO:8;
9) 16S rRNA gene of B. megaterium strain 4B referred in the present disclosure as SEQ ID NO:9;
10) 16S rRNA gene of B. pumulis strain R2E referred in the present disclosure as SEQ ID NO:10;
11) 16S rRNA gene of Bacillus aryabhattai strain R2A referred in the present disclosure as SEQ ID NO:11;
12) 16S rRNA gene of Bacillus sp. strain R2D referred in the present disclosure as SEQ ID NO:12;
13) 16S rRNA gene of B. megaterium strain 4C referred in the present disclosure as SEQ ID NO:13;
14) 16S rRNA gene of Enterobacter asburiae strain 6A referred in the present disclosure as SEQ ID NO: 14;
15) 16S rRNA gene of Massilia sp. strain RIC referred in the present disclosure as SEQ ID NO:15;
16) 16S rRNA gene of Ralstonia pickettii strain R3C referred in the present disclosure as SEQ ID NO:16; and
17) 16S IRNA gene of Enterobacter sp. Strain R4A referred in the present disclosure as SEQ ID NO:17.

### Example 2

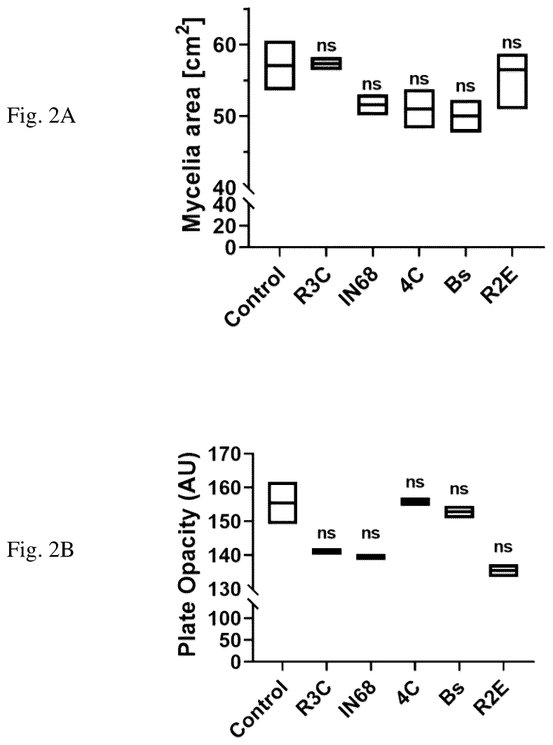

A standard co-inoculation technique was performed to determine the ability of said isolates to inhibit growth of a fungal phytopathogen, B. cinerea (Bcl16), and bacterial pathogen X. campestris pv. vesicatoria strain 85-10 (Xcv). B. cinerea was cultured on potato dextrose agar (PDA) at 22° C. for 5 days. A 5 mm diameter mycelial disc was cut from a 5-days-old B. cinerea colony and placed on one side of a dual media (PDA and LB media, 1:1) agar plates and incubated at 22° C. After 2 days of inoculation, tested bacteria were streaked at other ends of the plate and cultured at 28° C. for 5 days to examine antagonistic effects of these bacterial strains. The mycelia area (cm²) in colony radius compared to the control was calculated. 100 μl of X. campestris suspensions (around 10⁸ cells/ml) was mixed with LB Agar (0.6% agar) in pour plates. After solidification, 1 μl of bacterial suspensions (around 10⁸ cells/ml) was placed in the center of the agar plate and incubated at 28° C. for 24 h. After incubation, inhibition halos were measured in terms of plate opacity (AU). Plates without the bacteria served as control. Reference is now made to FIG. 2A graphically depicting B. cinerea (Bcl16) growth (as mycelia area in cm²) with the presence of selected microbial strains [viz., R. pickettii (R3C), P. aeruginosa (IN68), B. pumulis (R2E), B. megaterium (4C) and B. subtilis (Bs).] isolated from tomato leaves, and to FIG. 2B graphically depicting bacterial growth of X. campestris pv. vesicatoria strain 85-10 (as plate opacity) in the presence of said selected isolated strains. The results as depicted in both graphs show that the isolates from tomato leaves do not exhibit any significant direct antimicrobial activity against B. cinerea and X. campestris ex planta.

### Example 3

To assess the effect of said isolated bacterial strains on tomato growth, tomato seeds (S. lycopersicum, cv. M-82) were sown after surface sterilization (with 1.5% NaOCl for five minutes followed by three times rinsing with sterile water) in a tray containing the potting mixture. After germination, a single 21-day-old tomato seedling was transplanted into a pot (0.5 L, diameter=10 cm) containing green quality soil mix, Tuff soil, Israel, before inoculation of whole plants or detached tissues by pathogens. Pots were kept in the greenhouse at 25+2° C. and 16-h photoperiod. Isolated bacterial cultures along with two more isolates Pseudomonas putida (IN68) and B. subtilis (SB491) colonies from the plate culture (24 h old culture) were washed twice in sterile distilled water and then resuspended in sterile distilled water. The cell suspension was adjusted to an optical density of OD₆₀₀=1.00 (approximately equal to 1.0×10⁹ CFU ml⁻¹) using a spectrophotometer (Tecan). Soil drenching was carried out by pouring 10 ml of bacterial suspension (approximately equal to 1.0×10⁹ CFU ml⁻¹) into each pot twice a week. Plants treated with either sterile distilled water was served as mock controls. Plants from greenhouse experiments were sampled at 60 days after sowing of seeds to measure the growth parameters. Five plants in each treatment were used to measure number of inflorescences, plant height, tomato yield (g) per plant and harvest index (calculated as the ratio between the total fruit yield mass and total biomass).

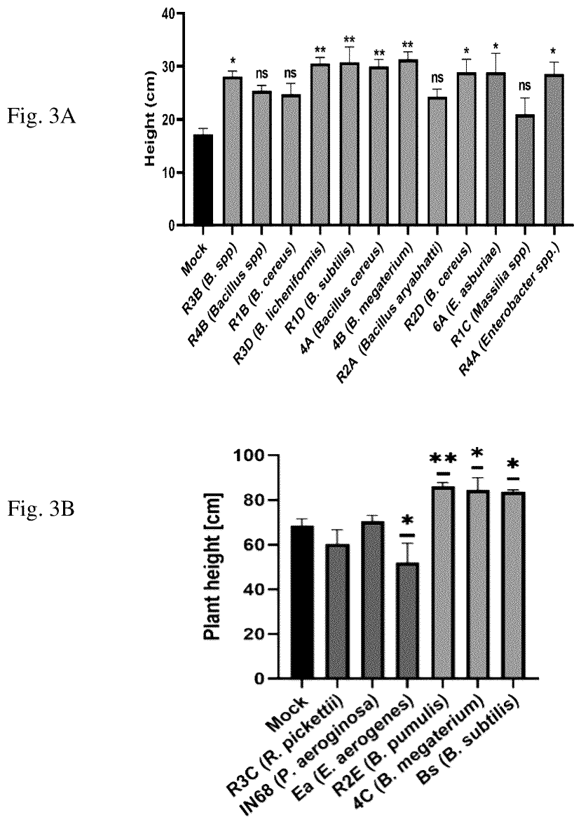

Reference is now made to FIG. 3A graphically depicting the statistically significant increase in tomato plants' height of treated plants compared to control plants. The tomato plants were treated with the following biological agents (bacterial isolates): Bacillus spp (R4B), B. cereus (RIB), B. licheninformis (R3D), B. subtilis (RID), B. megaterium (4B), Bacillus aryabhattai (R2A), E. asburiae (6A), Massilia spp (RIC) and Enterobacter spp. (R4A). Reference is also made to FIG. 3B graphically depicting the increase in plants' height of tomato plants treated with additional bacterial isolates: R. pickettii (R3C), P. aeruginosa (IN68), B. pumulis (R2E), B. megaterium (4C) and B. subtilis (Bs).

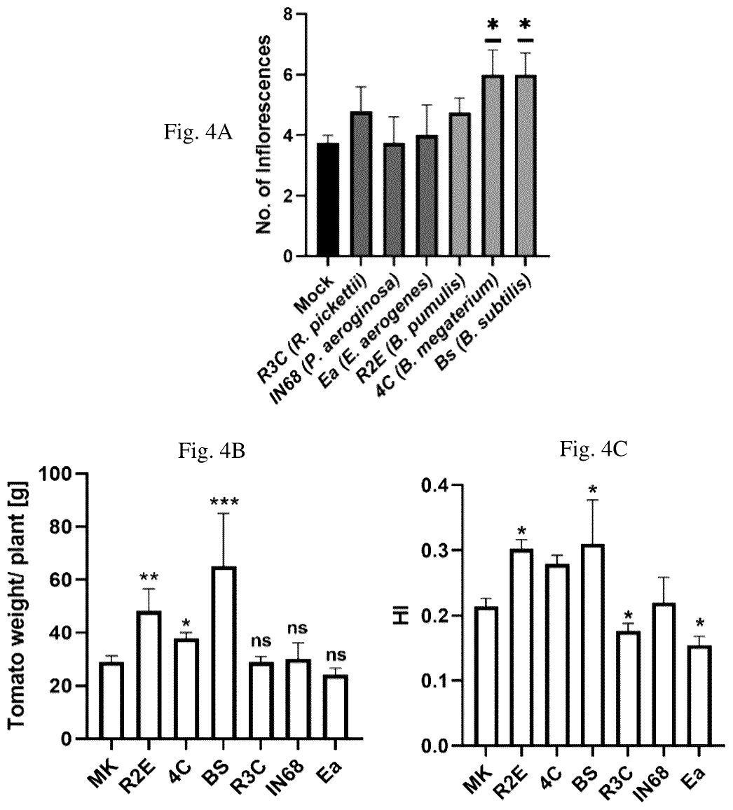

Reference is now made to FIG. 4A graphically depicting the number of inflorescences of tomato plants treated with the following bacterial isolates: R. pickettii (R3C), P. aeruginosa (IN68), B. pumulis (R2E), B. megaterium (4C) and B. subtilis (Bs), compared to mock.

Reference is now made to FIG. 4B graphically depicting tomato yield (as tomato weight/plant [gr]) of plants treated with the following bacterial isolates: R. pickettii (R3C), P. aeruginosa (IN68), B. pumulis (R2E), B. megaterium (4C) and B. subtilis (Bs), compared to mock.

Reference is now made to FIG. 4C graphically depicting the harvest index (ratio between the total fruit yield mass and total biomass) of plants treated with the following bacterial isolates: R. pickettii (R3C), P. aeruginosa (IN68), B. pumulis (R2E), B. megaterium (4C) and B. subtilis (Bs), compared to mock.

The results above clearly indicate that tomato plants treated with bacterial suspensions comprising bacteria that naturally inhabit the plants, exhibit improved traits, such as height, weight and number of inflorescences. Furthermore, the maximum increase in plants' height was recorded in tomato plants inoculated with B. pumulis (R2E). Results showed that plant inoculated with different species of Bacillus produced significantly higher tomato weight per plants (35-65 g/plant) as compared to mock (30 g/plant).

### Example 4

To evaluate the effect of the bacterial strains isolated from tomato leaves on plant resistance, tomato plants were treated with said strains and exposed to fungal and bacterial pathogens (B. cinereal and X. campestris, respectively).

B. cinerea (Bcl16) was cultured on potato dextrose agar (PDA) in petri dishes incubated at 22° ° C. The Bcl16 conidia were harvested from 14-day-old cultures and the suspension was then filtered through sterile cheesecloth. The concentration of conidia was determined using a haemocytometer under a light microscope, and adjusted to 10⁶ cells mL⁻¹ in solution (0.1% glucose and 0.1% K₂HPO₄) to the final conidial suspension. Each tomato leaflet was inoculated with two droplets of 10 μl spore suspension. Controls consisted of leaves treated with the above-mentioned solution without the presence of pathogenic agent. The area of the necrotic lesions on infected leaf tissue was measured 5-10 days post-inoculation using the ImageJ image processing software. For X. campestris pathogenicity assays, culture was grown in LB medium containing 100 mg L⁻¹ of rifampicin and 300 mg L⁻¹ of streptomycin, overnight at 28° C. Log phase bacterial cultures were harvested and re-suspended in 10 mM MgCl₂ at a final concentration of 10⁴ CFU mL-1 (OD₆₀₀=0.0002). The fourth leaf of 4-week-old tomato plants was vacuum-immersed with the bacterial suspensions. Three days after infiltration, three leaf discs of 0.9 cm diameter were sampled from at least four plants and ground in 1 ml of 10 mM MgCl₂. X. campestris CFU were determined by plating and counting the resulting colonies. Negative controls consisted of 10 mM MgCl₂ without pathogen inoculation.

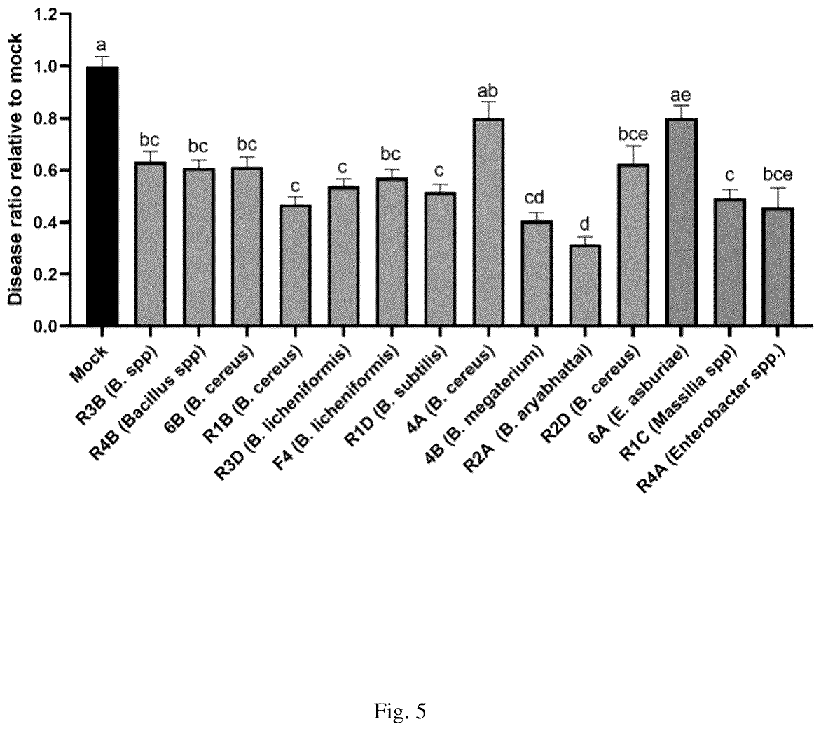

Reference is now made to FIG. 5 graphically depicting the effect of different bacterial isolates on B. cinerea Bcl16 disease development. As can be seen from the graph, tomato plants treated with the bacterial isolates (which function as biological control agents) manifested a reduced disease development compared to mock.

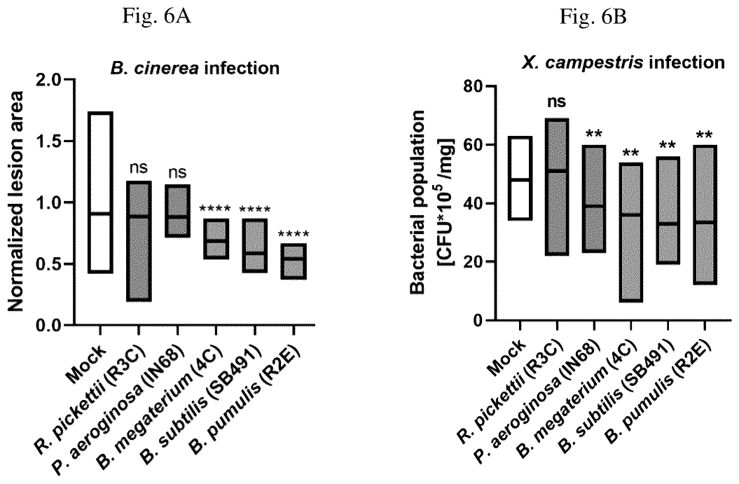

Reference is now made to FIG. 6A graphically depicting the effect of pre-treating tomato plants with selected BCAs [R. pickettii (R3C), P. aeruginosa (IN68), B. pumulis (R2E), B. megaterium (4C) and B. subtilis (Bs)] on fungal (B. cinerea Bcl16) lesion area.

Reference is also made to FIG. 6B graphically depicting the effect of pre-treating tomato plants with selected BCAs [R. pickettii (R3C), P. aeruginosa (IN68), B. pumulis (R2E), B. megaterium (4C) and B. subtilis (Bs)] on bacterial (X. campestris) disease symptoms, shown as bacterial population [CFUX10⁵/mg].

To conclude, the results above show that tomato plants inoculated with bacterial isolates (biological control agents) showed a significant decrease in fungal and bacterial infection (B. cinerea and X. campestris respectively), compared to the mock plants (plants without the bacterial isolates). Plants pre-treated with bio-agents and later infected with B. cinerea or Xcv showed less lesion area and number of colonies, respectively than mock plants.

### Example 5

As described in the preceding examples, exposing tomato seedlings to bacterial isolates which naturally inhabit the mature plants results in beneficial effects, such as improved traits and resistance to pathogenic fungi and bacteria. Further findings of the present application disclose that the exposure of plants to said beneficial isolates (biological control agents) activates their immune response, as manifested by ethylene production and reactive oxygen species (ROS) measurement.

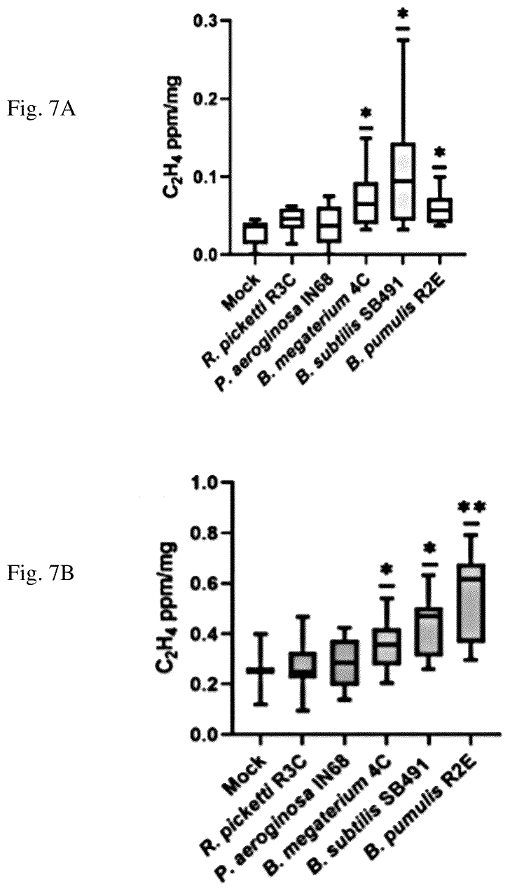

For ethylene production, leaf discs 0.9 cm in diameter were harvested from indicated treatments, and average weight was measured for each plant. Discs were washed in water for 1-2 h. Every six discs were sealed in a 10 mL flask containing 1 ml assay medium (with or without 1 μg ml⁻¹ EIX, ethylene induced xylanase) for 4 h at room temperature. Ethylene production was measured by gas chromatography (Varian 3350, Varian, California, USA). Reference is now made to FIG. 7A graphically depicting ethylene production (ppm/mg) in wounded tomato discs from plants treated with the following bacterial isolates: R. pickettii (R3C), P. aeruginosa (IN68), B. pumulis (R2E), B. megaterium (4C) and B. subtilis (Bs).

Reference is also made to FIG. 7B graphically depicting ethylene production (ppm/mg) in tomato discs from plants treated with the above-mentioned bacterial isolates and also triggered with EIX.

Ethylene production was dramatically increased in response to EIX and wounding in B. megaterium. B. subtilis and B. pumulis treated plants compared to mock.

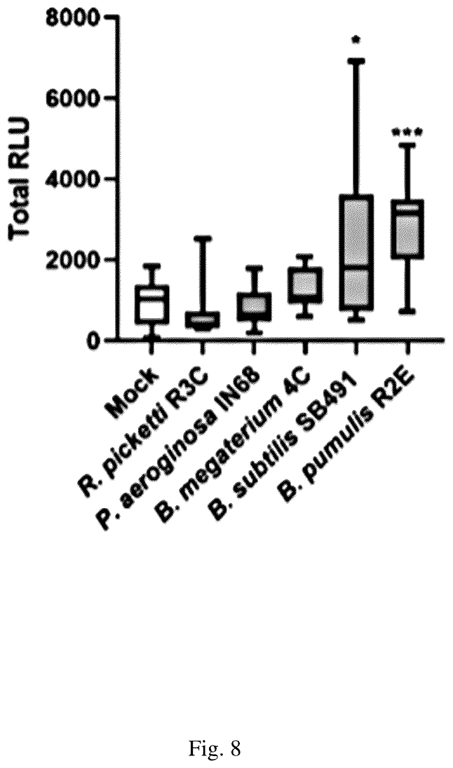

As for ROS measurement, tomato leaf discs of 0.5 cm in diameter were harvested from leaves 4-5 of 5-week old mock and bacterial treated tomato plants. Discs were floated in a white 96-well plate (SPL Life Sciences, Korea) containing 250 μl distilled water for 4-6 h at room temperature. Further, water was removed and a ROS measurement reaction containing 1 mM flg-22 or water was added. Light emission was measured for 30 minutes using a luminometer (Tecan Spark, Switzerland). Reference is now made to FIG. 8 graphically depicting ROS production measured immediately after flg-22 application for 35 minutes using the HRP-luminol method.

ROS measurements immediately after elicitation with flg-22 displayed enhancement of 200% in oxidative burst in B. pumulis treated plants, compared to that of the elicited mock. R3C and IN68 had no significant activity in the activation of plant defenses, whereas 4C had a lower effect on ROS production than the other bio-agents R2E and SB491. The overall enhancement of defense responses observed upon elicitation by EIX and flg-22 can be explained by the improved immunity of plants with Bacillus spp., underlying enhanced pathogen resistance.

### Example 6

In addition to evaluating ROS and ethylene production, gene expression was studied in tomato plants treated with biological control agents (the bacterial strains isolated from tomato leaves). RNA was isolated from ground leaves samples from plants treated with bacterial isolates using Tri reagent (Sigma-Aldrich) as per the manufacturer's recommendations. RNA concentrations were quantified and cDNA was then synthesized from 2 μg RNA in a 20 μL reaction using both reverse transcriptase and oligo(dT) primers provided with the cDNA Synthesis kit (Promega, United States). All defense genes were quantified using the Power SYBR Green Master Mix protocol (Life Technologies, Thermo Fisher, United States), using a Rotor-Gene Q machine (Qiagen) detection system. These genes are marker genes for jasmonic acid (JA), salicylic acid (SA) and ethylene (ET) signaling pathways, as shown in Table 1.

TABLE 1
Tomato defense-related and PRRs genes examined in this study and the specific
primers used in quantitative reverse transcriptase-polymerase chain reaction (qRT-PCR).

| Locus | Gene name | Primers (F) | (R) | References |
|---|---|---|---|---|
| Solyc01g111170 | Cyclophilin | TGAGTGGCTCAACGGAAAGC | CCAACAGCCTCTGCCTTCTTA | Pizarro et |
| | | | | al., 2020 |
| Solyc10g006580 | RPL8 | TGGAGGGCGTACTGAGAAAC | TCATAGCAACACCACGAACC | Harel et al., |
| | | | | 2014 |
| Solyc03g078400 | Actin | TGGTCGGAATGGGACAGAAG | CTCAGTCAGGAGAACAGGGT | Al-Abdallat |
| | | | | et al., 2015 |
| Solyc01g106620 | PR-la | CTGGTGCTGTGAAGATGTGG | TGACCCTAGCACAACCAAGA | Harel et al., |
| | | | | 2014 |
| Solyc05g051200 | ERF1 | ATTAGGGATTCAACGCGTAA | AGAGACCAAGGACCCCTCAT | Harel et al., |
| | | | | 2014 |
| Solyc07g049530 | ACO1 | GGGCTTCTTTGAGTTGGTGA | GCTTGAACAGCCTCAAGTCC | Harel et al., |
| | | | | 2014 |
| Solyc02g077370 | Pti 5 | GACATGGTGCGAGAGTATGG | CTGAAACAGAGGCGTTCACT | Harel et al., |
| | | | | 2014 |
| Solyc01g060020 | PR-2 | TCGAACAGGAGGAGGATCTG | TCCAGGCTTTCTCGGACTAC | Harel et al., |
| | (beta-1,3 | | | 2014 |
| | glucanase) | | | |
| Solyc10g055800 | PR-3 | AATGGTGGCCTAGAACGTGG | AGCTGAGTCCAACAGACTACA | Mehari et |
| | (Chitinase) | | | al., 2015 |
| Solyc02G070890 | FLS2 | GGGTTGGGGCAGTTATCCAA | GGTGGAATGGCACCTGAGAA | Pizarro et |
| | | | | al., 2020 |
| Solyc07g008620 | LeEIX1 | TCAGACACGCTTCCAAGTTGGTTC | ATTGGTAGGGACTAGTGGCAAAGC | Pizarro et |
| | | | | al., 2019 |

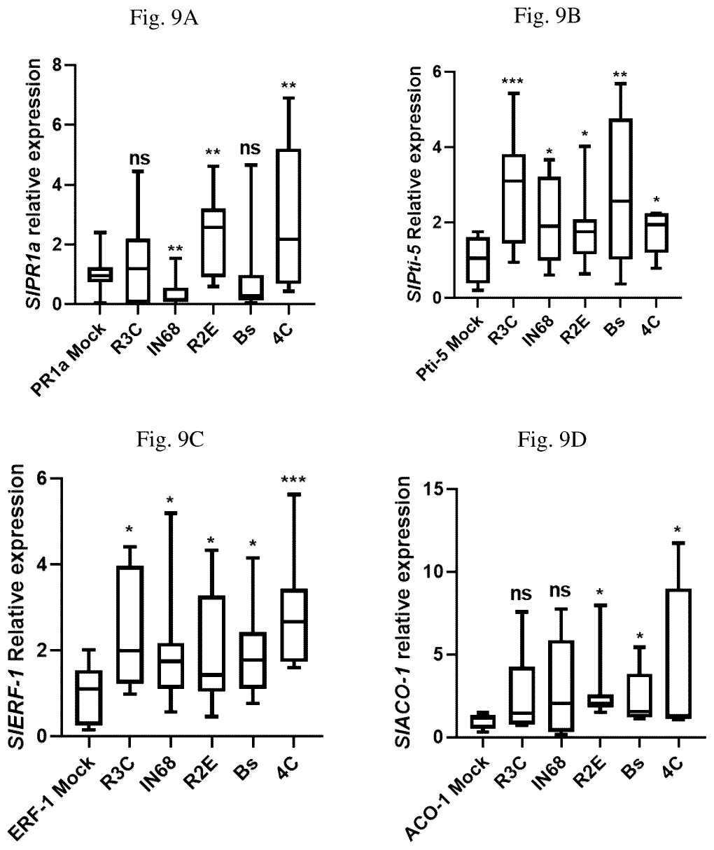

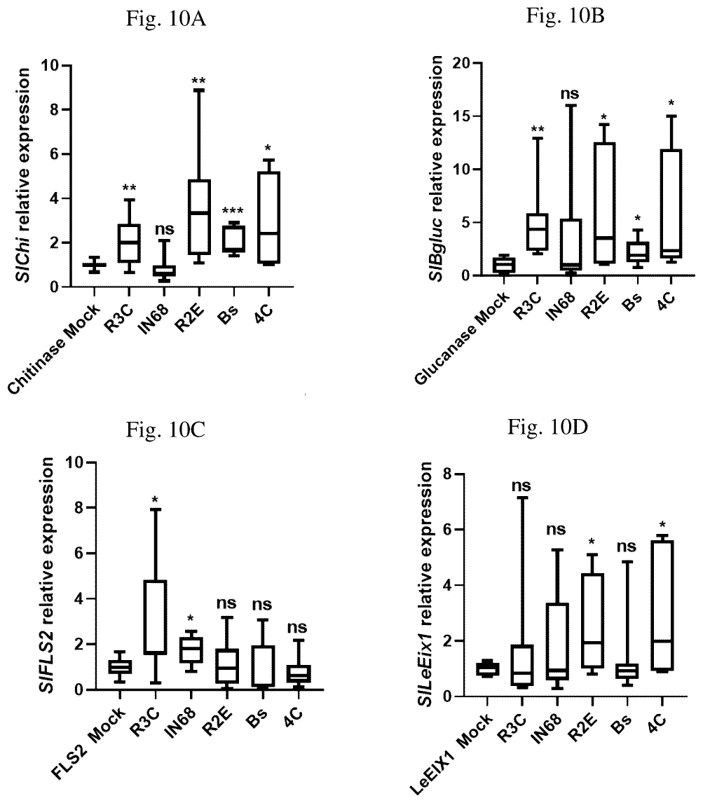

The ribosomal protein Sl-RPL8 (Solyc10g006580), Sl-cyclophilin (Solyc01g111170) and Sl-Actin (Solyc03g078400) were used as the housekeeping gene for normalization, and the cDNA was diluted to 1: 5000 before the amplification of this gene. Relative expression quantification was calculated using copy number method for gene expression experiments (see D'haene et al., 2010). Reference is now made to FIGS. 9-10, graphically depicting the relative expression of defense-related genes in tomato plants treated with the biological control agents of the present application.

FIG. 9A depicts relative expression of slPR1a, FIG. 9B depicts relative expression of slPti-5, FIG. 9C depicts relative expression of slERF-1, FIG. 9D depicts relative expression of slACO-1, FIG. 10A depicts relative expression of sICHI, FIG. 10B depicts relative expression of slBgluc, FIG. 10C depicts relative expression of sIFLS2, and FIG. 10D depicts relative expression of slLeEix1.

Application of bacterial bio-agents promoted the expression of defense-related genes in tomato plants, as compared to the mock. Specifically, mean transcript levels of genes PR-1a. PR-2 (B-glucanase), PR-3 (Chitinase), Pti5. ACO and ERF were higher after exposure to the bacterial bio-agents of the present application. PR-2 (glucanase) has been considered important in characterizing a beneficial microbes' ability to reduce disease (see Kamou et al., 2020).

The PR-3 gene family encodes for several types of endochitinases, and has mostly been reported to be induced by activation of JA/ET-signaling pathway in tomato. The PR-5 gene family encodes for thaumatin-like proteins and is involved in osmotic regulation of cells. Elevated transcript of ACO1. an ACC oxidase (ACO) that contributes in the final step of ET biosynthesis indicated the activation of the ET signaling pathway. ET is thought to signal ISR, in synergy with JA during root colonization by beneficial microorganisms. Such activation involves regulation of the ethylene-responsive factor 1 (ERF1) which is rapidly elicited by ET or JA and involves both signaling pathways and acts as a transcription factor for the regulation of genes responsive to various stresses. ET and JA synergy was verified by the increased expression levels of ERF1 after application of beneficial microbes. Another ET responsive factor that was elevated after bio-agent treatments was Pti5 (Pto-interacting protein 5) which is associated in resistance gene-mediated recognition of bacterial pathogens, independently of defense pathways (SA, ET/JA) and is assumed to activate SA-induced PR genes but not ET-regulated genes. The effect of the bacterial bio-agents of the present application was also studied on the expression of LeEIX1 (Ethylene-inducing xylanase) and Leucine-rich-repeat receptor like kinases-Flagellin sensitive 2 (LRR-RLKs-FLS2) in the tomato leaves. The EIX receptors (LeEix) belong to a superclade of Leucine-rich-repeat receptor proteins (LRR-RLPs), have been associated with the activation of defense responses signaling in plants. FLS2 recognizes bacterial flagellin and the flagellin-derived peptide flg22, has been linked with plant defense responses as well.

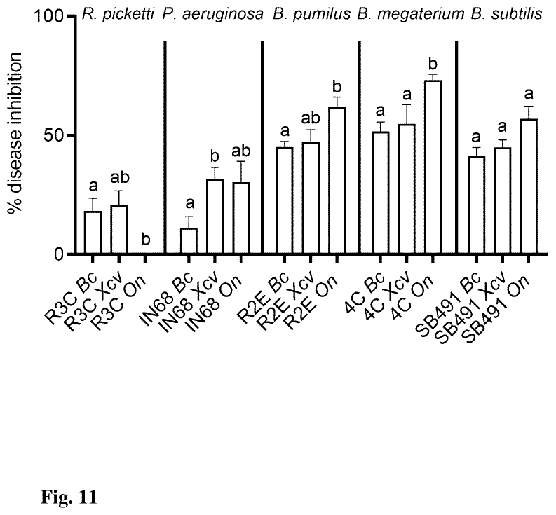

FIG. 11. Comparison of bacterial isolate activity across pathogen taxa. S. lycopersicum cv. M82 plants were pre-treated with indicated bacteria (OD₆₀₀=0.1), and separately challenged with the indicated pathogens 3 days after bacterial treatment. Bc: necrotrophic fungal pathogen Botrytis cinerea lesion area was measured 3 days after inoculation with B. cinerea (10⁶ spores mL⁻¹). Xcv: hemibiotrophic bacterial pathogen Xanthnomonas euvesicatoria growth (CFU) was measured 3 days after inoculation (10⁵ CFU mL⁻¹⁾. On: biotrophic fungal pathogen Oidium neolycopersici disease area was measured 7 days after inoculation (10⁵ spores mL⁻¹).

Conclusion: Bacilli Isolates R2E and 4C have High and Similar Activity Against Three Different Pathogen Classes.

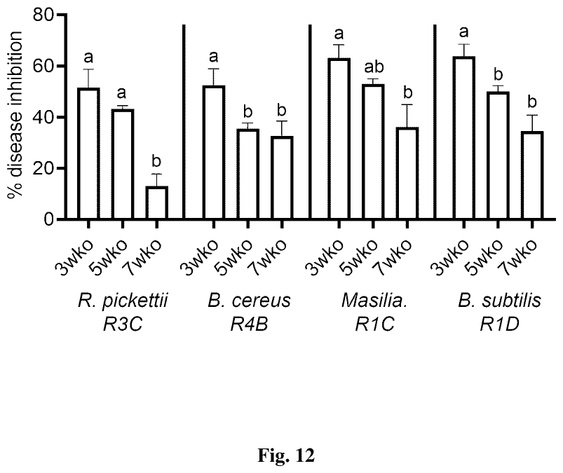

FIG. 12. Comparison of bacterial isolate activity across plant developmental ages. S. lycopersicum cv. M82 plants of indicated ages were pre-treated with indicated bacteria (OD₆₀₀=0.1). and challenged with B. cinerea 3 days later. Lesion area was measured 3 days after inoculation with B. cinerea (10⁶ spores mL⁻¹).

Conclusion: Out of the Ages Examined, 3 Week Old Plants Respond Best to Bacterial Treatment with Certain Bacterial Isolates.

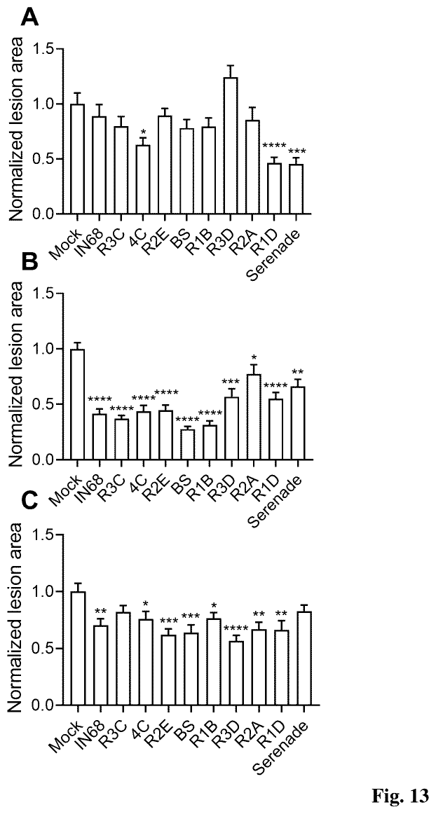

FIG. 13. Comparison of bacterial isolate activity across plant developmental ages. S. lycopersicum cv. M82 seeds or plants were pre-treated with indicated bacteria (OD₆₀₀=0.1). and challenged with B. cinerea. Lesion area was measured 3 days after inoculation with B. cinerea (10⁶ spores mL⁻¹).
A: Seeds were soaked in bacterial isolates, and disease was examined 3 weeks after germination.
B: 3-week-old plants were treated with bacterial isolates, and disease was examined 3 days after treatment.
C: 6-week-old plants were treated with bacterial isolates, and disease was examined 3 days after treatment.
   Conclusions: (1) 3-Week-Old Plants Respond Best to Bacterial Treatment with Certain Bacterial Isolates; (2) Seed Coatings can be Effective in Reducing Disease; (3) Treating Mature Plants with the Bacterial Isolates is Effective

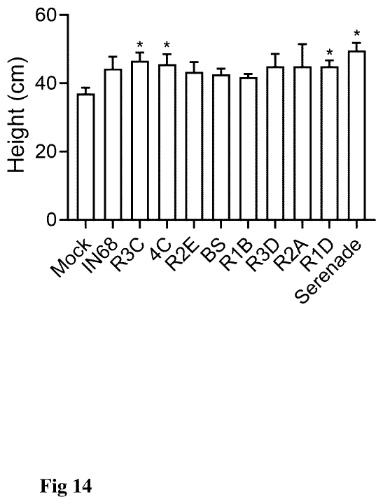

FIG. 14. Effect of seed coating with bacterial isolates on plant development. S. lycopersicum cv. M82 seeds were soaked with indicated bacteria (OD₆₀₀=0.1) prior to germination. Plant were subsequently grown for 50 days and height was measured.

Conclusions: Seed coatings with certain bacterial isolates can be effective in improving plant growth and development.

SEQUENCE LISTING
OrganismName: Bacillus sp. strain R3B
PreSequenceString:
Sequence 1
TCCTTGTCACTTAGGCGGCTAGCTCCTTACGGTTACTCCACCGAC
TTCGGGTGTTACAAACTCTCGTGGTGTGACGGGCGGTGTGTACAA
GGCCCGGGAACGTATTCACCGCGGCATGCTGATCCGCGATTACTA
GCGATTCCAGCTTCATGTAGGCGAGTTGCAGCCTACAATCCGAAC
TGAGAATGGTTTTATGGGATTGGCTTGACCTCGCGGTCTTGCAGC
CCTTTGTACCATCCATTGTAGCACGTGTGTAGCCCAGGTCATAAG
GGGCATGATGATTTGACGTCATCCCCACCTTCCTCCGGTTTGTCA
CCGGCAGTCACCTTAGAGTGCCCAACTAAATGCTGGCAACTAAGA
TCAAGGGTTGCGCTCGTTGCGGGACTTAACCCAACATCTCACGAC
ACGAGCTGACGACAACCATGCACCACCTGTCACTCTGTCCCCCGA
AGGGGAACGCTCTATCTCTAGAGTTGTCAGAGGATGTCAAGACCT
GGTAAGGTTCTTCGCGTTGCTTCGAATTAAACCACATGCTCCACC
GCTTGTGCGGGCCCCCGTCAATTCCTTTGAGTTTCAGTCTTGCGA
CCGTACTCCCCAGGCGGAGTGCTTAATGCGTTAGCTGCAGCACTA
AAGGGCGGAAACCCTCTAACACTTAGCACTCATCGTTTACGGCGT
GGACTACCAGGGTATCTAATCCTGTTTGCTCCCCACGCTTTCGCG
CCTCAGCGTCAGTTACAGACCAAAAAGCCGCCTTCGCCACTGGTG
TTCCTCCACATCTCTACGCATTTCACCGCTACACGTGGAATTCCG
CTTTTCTCTTCTGCACTCAAGTTCCCCAGTTTCCAATGACCCTCC
ACGGTTGAGCCGTGGGCTTTCACATCAGACTTAAGAAACCGCCTG
CGCGCGCTTTACGCCCAATAATTCCGGATAACGCTTGCCACCTAC
GTATTACCGCGGCTGCTGGCACGTAGTTAGCCGTGGCTTTCTGGT
TAGGTACCGTCAAGGTACAAGCAGTTACTCTTGTACTTGTTCTTC
CCTAACAACAGAATTTTACGACCCGAAAGCCTTCATCACTCAAGC
GGCGTTGCTCCGTCAGACTTTCGTCCATTGCGGAAGATTCCCTAC
TGCTGCCCCCCCTAAGAATCTGGGGCCGGGTCTCAATCCCCCTGT
GGGCCAAAACCCCCTCCAGGTCGCCCATGAAACCTTTGCCTTGGT
GAACCGTTACCTCCCAACTAACTTATGGCCCCCCGCGCCCCCCTT
TAATGTATAACCCAAACCAATTTTTATTAACTCCCCCGAAAGGGA
AAAATCAATTCCGGTTTAATTTCGGGTTTCCCAAATTTTCCCACC
TTTAAAGGAGGGTTGCCCCACGTTTATCACCCTTCCCCCCTCATT
CTAAAAAAAAACTTTAAATTTTTCGAATTGGTAAGGCTGGCCCCC
CCCGGGCGGGACTCACTCAGGTAAAAAAAAAAAAATTATGAACCC
TAAAAAAATAAACCCCCGGGCCACCAAAATATTAAAGAACCCAAA
AACAAAAAAATTGAACCGTTTCCCCTGGCCCTGTTTGTGTGTTGT
TGATGCGGGCCGGTGTAAACGGGTATTTGGTGACCCCTGGGCTTT
GGTGGCCGCTGTAATGGCGTGATGTTTGTTTTTTGGTGTTT
Type: DNA
Length: 1661
OrganismName: Bacillus sp. strain R4B
PreSequence String:
Sequence 2
GAATGGGGGGGGGTTGCTATAATGCAGTCGAGCGATCGATGGTAG
CTTGGTCCTTCAGATTATCGGGGGATGGGTGAGTGTGCGTGGGGG
CCCGGCCTGTAAGACAGGGACAACTTCGGGAAACCGGAGCTAATA
CCGGATATTTTCTTTTCTCACATGAGATAAAATGGAGAGACGGTT
TATGGTCTCGGGGGACCCGTTGCCCCCGCGGCGTTGGGGATTGGT
TGGAGGCCAATGGCACCAAGGCGACGATATGGTTTAGGGGGGGGG
GGGGGTTGGGGCTTACAGGGACCCCGAACCCTCCCACAGGCGCAT
TCGGTGGAGTCTCCCCCAGAAATCCCGCAAAGGACAAGAATCTCC
GGGGGCGAGGCCCCGTGTTAAATAAAAAAACTCGGGTTTTGAAAA
AAAAAGTCGGGGAGAAAAGGCGGGCCATTAAATGGAGGGAACCCT
AAGGGACCTCCTAAAAAAAGCCGGGCGCAGAAGTTGGCCTGGTCC
CCCGTATTTCTCTTTATGTATACCCTTTTTGGCCGGTATTTGGTG
GGTTAATAATGACGCCCGGGGGTTCCCCCCCCCCCTCCCCGAGGA
AGCCCCCCCGCTTGCTGCCCGTGGGGGGGCTTTTGGGAAAAATGG
GAACATTTCCTTCTCTCACAAAAAAATGTGATATTTTTTCGGGGG
GGGGGGAAAAAAGGTGGAATTTTTTCGGTGAGTCCCCGAACTTCA
GGCACGAATCGGGGGGAGAGAACAAAATAGCTGCGCCTCCACTGC
TTTTCGCAAAAGATGACGGCAGTTGAAGGGCGACTCCCGGCTTCT
CACGTCTAACTTCGCAGACAAACACTCAGCCCGCGGGTGGGCTCC
CGCGGGGTCTTAACCCGAAGTATTTTAAGCATCTCACTGGGAGCA
TCCGTGCGCGTGGGTATAACGAATAGAATCCCATAAATCTGGGTA
GGCCTGGGGTATGTACTTAATCGTAGGATAGCTGTTCGCTTGCGG
TTTTAGAATGGAGGGAGGAGCTACGGTCGTCCCGTCCCTAGCAAT
CAAATGTTAGCCGTCCAGAAAGCGGACATTATTCACCACAGAAAT
TTCTATCTTTCGTCAGTACGTCTTTTCGTCCCTCACGTAACACGT
CTTCAGGTCCGAAAGAAGATACCGAGGGAGGAGCGCCTCCACTCG
CATTGATG
Type: DNA
Length: 1178
OrganismName: B. cereus strain 6B
PreSequenceString:
Sequence 3
CCCCTCCCGGGTTTTTTTTCTTTTTTTTTGTTTTAACCCCCCCCC
CCCCCCCCCTTACCTAAGTGGTCTCTCGGACAGAGGAGCACGGGC
TTTACCGATTGGGAAAGTTTAAGCTTTTTCTAAGATTGGGGGACG
GGGAACACCGGGTTACCGCCCAAAGGGTGGAAATTCGGGAACCGG
GGTTAATCCGGATACCTTTTGAACCGCATGTTTGAAATGAAAGGC
GGTTTGGCTTTCATTTATGGAGGACCCGGCTCGCATTAGCTAGTG
GGGAGGTAACGGTTCACCAAGGCAACGATGGGTAGCCGACCTGAG
AGGTGATCGGCCACACTGGGACTGAGACACGGCCCAGATTCTTAC
GGGAGGCAGCAGTAGGGATTTTCCGCAATGGACGAAAGTCTGACG
GAGCAACGCCGCGTGAATGATGAAGGCTTTCGGGTTGTAAAACTT
TGTTGTTAGGGAAGAACAAGTGCTAGTTGAATAAGCTGGCACCTT
GACGGTACCTAACCAGAAAGCCACGGCTAACTACGTGCCAGCAGC
CGCGGTAATACGTAGGTGGCAAGCGTTATCCGGAATTATTGGGCG
TAAAGCGCGCGCAGGTGGTTTCTTAAGTCTGATGTGAAAGCCCAC
GGCTCAACCGTGGAGGGTCATTGGAAACTGGGAGACTTGAGTGCA
GAAGAGGAAAGTGGAATTCCATGTGTAGCGGTGAAATGCGTAGAG
ATATGGAGGAACACCAGTGGCGAAGGCGACTTTCTGGTCTGTAAC
TGACACTGAGGCGCGAAAGCGTGGGGAGCAAACAGGATTAGATAC
CCTGGTAGTCCACGCCGTAAACGATGAGTGCTAAGTGTTAGAGGG
TTTCCGCCCTTTAGTGCTGAAGTTAACGCATTAAGCACTCCGCCT
GGGGAGTACGGCCGCAAGGCTGAAACTCAAAGGAATTGACGGGGG
CCCGCACAAGCGGTGGAGCATGTGGTTTAATTCGAAGCAACGCGA
AGAACCTTACCAGGTCTTGACATCCTCTGAAAACCCTAGAGATAG
GGCTTCTCCTTCGGGAGCAGAGTGACAGGTGGTGCATGGTTGTCG
TCAGCTCGTGTCGTGAGATGTTGGGTTAAGTCCCGCAACGAGCGC
AACCCTTGATCTTAGTTGCCATCATTAAGTTGGGCACTCTAAGGT
GACTGCCGGTGACAAACCGGAGGAAGGTGGGGATGACGTCAAATC
ATCATGCCCCTTATGACCTGGGCTACACACGTGCTACAATGGACG
GTACAAAGAGCTGCAAGACCGCGAGGTGGAGCTAATCTCATAAAA
CCGTTCTCAGTTCGGATTGTAGGCTGCAACTCGCCTACATGAAGC
TGGAATCGCTAGTAATCGCGGATCAGCATGCCGCGGTGAATACGT
TCCCGGGCCTTGTACACACCGCCCGTCACACCACGAGAGTTTGTA
ACACCCGAAGTCGGTGGGGTAACCTTTTTGGAGCCAGCCGCCTAA
GTGACAGTAATTGA
Type: DNA
Length: 1500
OrganismName: B. cereus strain RIB
PreSequenceString:
Sequence 4
GCGCATGGCGGGTGCTATACTGCAAGTCGAGCGAATGGATTAAGA
GCTTGCTCTTATGAAGTTAGCGGCGGACGGGTGAGTAACACGTGG
GTAACCTGCCCATAAGACTGGGATAACTCCGGGAAACCGGGGCTA
ATACCGGATAACATTTTGAACCGCATGGTTCGAAATTGAAAGGCG
GCTTCGGCTGTCACTTATGGATGGACCCGCGTCGCATTAGCTAGT
TGGTGAGGTAACGGCTCACCAAGGCAACGATGCGTAGCCGACCTG
AGAGGGTGATCGGCCACACTGGGACTGAGACACGGCCCAGACTCC
TACGGGAGGCAGCAGTAGGGAATCTTCCGCAATGGACGAAAGTCT
GACGGAGCAACGCCGCGTGAGTGATGAAGGCTTTCGGGTCGTAAA
ACTCTGTTGTTAGGGAAGAACAAGTGCTAGTTGAATAAGCTGGCA
CCTTGACGGTACCTAACCAGAAAGCCACGGCTAACTACGTGCCAG
CAGCCGCGGTAATACGTAGGTGGCAAGCGTTATCCGGAATTATTG
GGCGTAAAGCGCGCGCAGGTGGTTTCTTAAGTCTGATGTGAAAGC
CCACGGCTCAACCGTGGAGGGTCATTGGAAACTGGGAGACTTGAG
TGCAGAAGAGGAAAGTGGAATTCCATGTGTAGCGGTGAAATGCGT
AGAGATATGGAGGAACACCAGTGGCGAAGGCGACTTTCTGGTCTG
TAACTGACACTGAGGCGCGAAAGCGTGGGGAGCAAACAGGATTAG
ATACCCTGGTAGTCCACGCCGTAAACGATGAGTGCTAAGTGTTAG
AGGGTTTCCGCCCTTTAGTGCTGAAGTTAACGCATTAAGCACTCC
GCCTGGGGAGTACGGCCGCAAGGCTGAAACTCAAAGGAATTGACG
GGGGCCCGCACAAGCGGTGGAGCATGTGGTTTAATTCGAAGCACG
CGAAGACCTTACCAGGTCTTGACTCCTCTGAAACCCTAGAGATAG
GCTTCTCTTCGGAACAAATGACAGTGGTGCTGTTGCCTCACTCGG
GCCGGAATGTGGGTAAGTCCGCACGAGGCACCTTGACTTTTTGCC
GCATTATTTGGGACTTCAGGGACGCCGTGAAAACGGGGGAAGGGG
GGGTACTCCACTTTTAGCCCTTAAACGGGGTAAAAGGGTTAAAAG
AGGGAAAAAACTCCAAACCCCGGTTGAAAATTCTTAAACCTTTTT
TTTGAATAGGGCCCCCCCCCCCCAAGGGGAACCTTAAAACGAAAA
AAACCCCGGAAATTCCCCCCTTCCCCCCCGCCCCCCAAATTAACT
CCAACCTTGGACCTTCCCCCCCAGGATTAGGTTACATTTACCGAA
ACCCCTACGATTGAAGAATTGATGATAGAAAAATTAAGTTTGGGT
GGTTT
Type: DNA
Length: 1400
OrganismName: B. licheniformis strain R3D
PreSequenceString:
Sequence 5
CCCCTCATAATTAACGCAGACCACCACCAAGAATAATACCCTTCT
CCTCCCTTTTTTGATTTTTTTTTTTTAATGGCCGGCCCGGGCACT
ACAAGGGAAAGGTTTCCGAGGGGGGGGGAAAAGGGTACCCCTAAA
GGGAATCGGAACGGGTTACCGTTTTTTACCCTGTTAAAAAAGGGT
TTGTTCATTAGGGGCCGGGCCTTATTGTGGGGAACGCTCCCAGGC
ACGTGGTGCCACTGGGGGTATGCCCCCGGGATGAACCGCCCAATT
CTAGGGAGCGCGTAGGATTTCGCAAGGAGAAGTCGACGAGAACGC
GCTGATGAGAAGTTGACGAGCACGCCGCGTGAGTGATGAAGTTTT
CGATCGTAAACTCTGTGTAGGGAGACAGTACCGTCGATAGGGCGG
CACCTGACGGTACCTAACCAGAAAGCCACGGCTAACTACGTGCCA
GCAGCCGCGGTAATACGTAGGTGGCAAGCGTGTCCGGAATTATTG
GGCGTAAAGCGCGCGCAGGCGGTTTCTTAAGTCTGATGTGAAAGC
CCCCGGCTCAACCGGGGAGGGTCATTGGAAACTGGGGAACTTGAG
TGCAGAAGAGGAGAGTGGAATTCCACGTGTAGCGGTGAAATGCGT
AGAGATGTGGAGGAACACCAGTGGCGAAGGCGACTCTCTGGTCTG
TAACTGACGCTGAGGCGCGAAAGCGTGGGGAGCGAACAGGATTAG
ATACCCTGGTAGTCCACGCCGTAAACGATGAGTGCTAAGTGTTAG
AGGGTTTCCGCCCTTTAGTGCTGCAGCAAACGCATTAAGCACTCC
GCCTGGGGAGTACGGTCGCAAGACTGAAACTCAAAGGAATTGACG
GGGGCCCGCACAAGCGGTGGAGCATGTGGTTTAATTCGAAGCAAC
GCGAAGAACCTTACCAGGTCTTGACATCCTCTGACAACCCTAGAG
ATAGGGCTTCCCCTTCGGGGGCAGAGTGACAGGTGGTGCATGGTT
GTCGTCAGCTCGTGTCGTGAGATGTTGGGTTAAGTCCCGCAACGA
GCGCAACCCTTGATCTTAGTTGCCAGCATTCAGTTGGGCACTCTA
AGGTGACTGCCGGTGACAAACCGGAGGAAGGTGGGGATGACGTCA
AATCATCATGCCCCTTATGACCTGGGCTACACACGTGCTACAATG
GGCAGAACAAAGGGCAGCGAAGCCGCGAGGCTAAGCCAATCCCAC
AAATCTGTTCTCAGTTCGGATCGCAGTCTGCAACTCGACTGCGTG
AAGCTGGAATCGCTAGTAATCGCGGATCAGCATGCCGCGGTGAAT
ACGTTCCCGGGCCTTGTACACACCGCCCGTCACACCACGAGAGTT
TGTAACACCCGAAGTCGGTGAGGTAACCTTTGGAGCCAGCCGCCG
AAGTGCGAGTTGA
Type: DNA
Length: 1408
OrganismName: B. licheniformis strain F4 27F
PreSequence String:
Sequence 6
CACATCTCCGCCTCTTCATGGGAGTCCCAAGGTCTGACGGAGCAC
CGCGCTGGGATGGATGAAGGTTTTCGGTTCGTAAAACTCTGTTGT
TAGGAGAACAAGTACGTCGATAGGGCCGTACCTTGACGGTACCTA
ACCAGAAAGCACGGCTAACTACGTGCCAGCAGCCGCGGTAATACG
TAGGTGGCAAGCGTGTCCGGAATTATTGGGCGTAAAGCGCGCGCA
GGCGGTTTCTTAAGTCTGATGTGAAAGCCCCGGCTCAACCGGGGA
GGGTCATTGGAAACTGGGGAACTTGAGTGCAGAAGAGGAGAGTGG
AATTCCACGTGTAGCGGTGAAATGCGTAGAGATGTGGAGGAACAC
CAGTGGCGAAGGCGACTCTCTGGTCTGTAACTGACGCTGAGGCGC
GAAAGCGTGGGGAGCGAACAGGATTAGATACCCTGGTAGTCCACG
CCGTAAACGATGAGTGCTAAGTGTTAGAGGGTTTCCGCCCTTTAG
TGCTGCAGCAAACGCATTAAGCACTCCGCCTGGGGAGTACGGTCG
CAAGACTGAAACTCAAAGGAATTGACGGGGGCCCGCACAAGCGGT
GGAGCATGTGGTTTAATTCGAAGCAACGCGAAGAACCTTACCAGG
TCTTGACATCCTCTGACAACCCTAGAGATAGGGCTTCCCCTTCGG
GGGCAGAGTGACAGGTGGTGCATGGTTGTCGTCAGCTCGTGTCGT
GAGATGTTGGGTTAAGTCCCGCAACGAGCGCAACCCTTGATCTTA
GTTGCCAGCATTCAGTTGGGCACTCTAAGGTGACTGCCGGTGACA
AACCGGAGGAAGGTGGGGATGACGTCAAATCATCATGCCCCTTAT
GACCTGGGCTACACACGTGCTACAATGGGCAGAACAAAGGGCAGC
GAAGCCGCGAGGCTAAGCCAATCCCACAAATCTGTTCTCAGTTCG
GATCGCAGTCTGCAACTCGACTGCGTGAAGCTGGAATCGCTAGTA
ATCGCGGATCAGCATGCCGCGGTGAATACGTTCCCGGGCCTTGTA
CACACCGCCCGTCACACCACGAGAGTCCTAATCTCTCCTCAACGG
CTTGTCGTTAAGTTTCCCTGCGAAATACCCCGCGCTCCCCCGGAA
TTGGCAGTTCTCCCTCGGGCTGACACTCCCTGGAGCAGCGGTTCC
TGTAAAATCGCTCCCCTCTAAGGCCCCGCGTTTTGTGTTATAACC
ACCCTCTTCGCCGGGTTCTGCTCCGTGACGGTCCTAAGTCCTTCC
CGCGCCGCCCGTGCCCTCCGCCAACGAAATACTTTCCCTCGTAGC
GGCCGCCCCGGCCCAAA
Type: DNA
Length: 1322
OrganismName: B. subtilis strain RID
PreSequence String:
Sequence 7
TCACTCCTGTCACTTCGGCGGCTGGCTCCATAAAGGTTACCTCAC
CGACTTCGGGTGTTACAAACTCTCGTGGTGTGACGGGCGGTGTGT
ACAAGGCCCGGGAACGTATTCACCGCGGCATGCTGATCCGCGATT
ACTAGCGATTCCAGCTTCACGCAGTCGAGTTGCAGACTGCGATCC
GAACTGAGAACAGATTTGTGGGATTGGCTTAACCTCGCGGTTTCG
CTGCCCTTTGTTCTGTCCATTGTAGCACGTGTGTAGCCCAGGTCA
TAAGGGGCATGATGATTTGACGTCATCCCCACCTTCCTCCGGTTT
GTCACCGGCAGTCACCTTAGAGTGCCCAACTGAATGCTGGCAACT
AAGATCAAGGGTTGCGCTCGTTGCGGGACTTAACCCAACATCTCA
CGACACGAGCTGACGACAACCATGCACCACCTGTCACTCTGCCCC
CGAAGGGGACGTCCTATCTCTAGGATTGTCAGAGGATGTCAAGAC
CTGGTAAGGTTCTTCGCGTTGCTTCGAATTAAACCACATGCTCCA
CCGCTTGTGCGGGCCCCCGTCAATTCCTTTGAGTTTCAGTCTTGC
GACCGTACTCCCCAGGCGGAGTGCTTAATGCGTTAGCTGCAGCAC
TAAGGGGCGGAAACCCCCTAACACTTAGCACTCATCGTTTACGGC
GTGGACTACCAGGGTATCTAATCCTGTTCGCTCCCCACGCTTTCG
CTCCTCAGCGTCAGTTACAGACCAGAGAGTCGCCTTCGCCACTGG
TGTTCCTCCACATCTCTACGCATTTCACCGCTACACGTGGAATTC
CACTCTCCTCTTCTGCACTCAAGTTCCCCAGTTTCCAATGACCCT
CCCCGGTTGAGCCGGGGGCTTTCACATCAGACTTAAGGAACCGCC
TGCGAGCCCTTTACGCCCAATAATTCCGGACAACGCTGGCCACCT
ACGTATTACCGCGGCTGCTGGCACGTAGTTAGCCGTGGCTTTCTG
GTTAGGTACCGTCAAGGTACCGCCCTATTCGAACGGTACTTGTTC
TTCCCTAACAACAGAGCTTTACGATCCGAAAACCTTCATCACTCA
AGCGGCGTTGCTCCGTCAGACTTTCTTCCATTGCGGAAAAATTCC
CTACTGCTGCCTCCCCCTAAGAAATCTGGGGCCGTGTCTCAATCC
CCCTTGTGGGCCAACACCCTCTCCAGGGCGGCCAACCACCCCTTT
CCCTTGTGGAAGCCATTACTCCACCAACAAATTAAAGGGCCGCGG
GGCCTCCTTTAATTGGTATCCAAACCACCCTTTTTTTTTTTAACC
AAGGGGGTTAAAAAAAAAACCCGGGTATAAACCCCCGTTTCCCGG
GTATTTCCACCTTTTAAGGGGGGGTGACCCCGGTTTATCCCCCTC
CCCCCCCTAAAAAGGAAAGATTCAACTTCCCCCCAATCCTTAAAG
CTTGGGCCCCCGCCGGGTGCCTACACGGAAAAAAAAAAAAAAAGA
CAAATATAACTAAAATAACGAACCCAGAAAATAAGGGAGAAAAAG
TAAAATAAGATATTAAAGCCCCCCCCCGGGGGGAGAAATTGGAAA
CCCTTTAAAGTTTTGTTAAAGGGGGTAATATAAAAAAC
Type: DNA
Length: 1613
OrganismName: B. cereus strain 4A
PreSequenceString:
Sequence 8
GTGCTAGCGGGTCCTATACATGCAAGTCGAGCGAATGGATTGAGA
GCTTGCTCTCAAGAAGTTAGCGGCGGACGGGTGAGTAACACGTGG
GTAACCTGCCCATAAGACTGGGATAACTCCGGGAAACCGGGGCTA
ATACCGGATAACATTTTGAACTGCATGGTTCGAAATTGAAAGGCG
GCTTCGGCTGTCACTTATGGATGGACCCGCGTCGCATTAGCTAGT
TGGTGAGGTAACGGCTCACCAAGGCAACGATGCGTAGCCGACCTG
AGAGGGTGATCGGCCACACTGGGACTGAGACACGGCCCAGACTCC
TACGGGAGGCAGCAGTAGGGAATCTTCCGCAATGGACGAAAGTCT
GACGGAGCAACGCCGCGTGAGTGATGAAGGCTTTCGGGTCGTAAA
ACTCTGTTGTTAGGGAAGAACAAGTGCTAGTTGAATAAGCTGGCA
CCTTGACGGTACCTAACCAGAAAGCCACGGCTAACTACGTGCCAG
CAGCCGCGGTAATACGTAGGTGGCAAGCGTTATCCGGAATTATTG
GGCGTAAAGCGCGCGCAGGTGGTTTCTTAAGTCTGATGTGAAAGC
CCACGGCTCAACCGTGGAGGGTCATTGGAAACTGGGAGACTTGAG
TGCAGAAGAGGAAAGTGGAATTCCATGTGTAGCGGTGAAATGCGT
AGAGATATGGAGGAACACCAGTGGCGAAGGCGACTTTCTGGTCTG
TAACTGACACTGAGGCGCGAAAGCGTGGGGAGCAAACAGGATTAG
ATACCCTGGTAGTCCACGCCGTAAACGATGAGTGCTAAGTGTTAG
AGGGTTTCCGCCCTTTAGTGCTGAAGTTAACGCATTAAGCACTCC
GCCTGGGGAGTACGGCCGCAAGGCTGAAACTCAAAGGAATTGACG
GGGGCCCGCACAAGCGGTGGAGCATGTGGTTTAATTCGAAGCACC
CCAAGAACCTTACCAGGTCTTGACATCCTCTGAAAACCCTAGAGA
TAGGGCTTCTCCTTCGGGAGCAAAGTGACAGGTGGTGCATGCTTG
TCTTCATCTCTTGCCCTGAAATGTGGGGTAAGTCCCCCAACAACG
CCACCCTTGATTTACTTGCAACATAAGTTGGGACCCAAAGGGAAT
GCCGGGGACACCCGGAAGAAGCGGGGATGACCTTATACTACATGC
TATATAGTACTGGGCTCACCCGGGTTCAATGGCCGGTCAACAATT
GCCGACCCAAGGTGATCAATCCCTTACCGGTCAATTCCGAGGGGG
GCTAATTCCATTGAATTGAAACCTTTAACCGGAAACCACCCTTTG
GAAACCTTCCGGCCGTTAACCGACCCGGCCGGAATCTTGAACCCT
TACCGGGGGGCTTTTTGACCGGCCTTGGCCTTTTTGGGGGTAAGG
GCGGAAACGAAACGGAAAGCCCAAAAAATATACATATATAATATT
CCAATAAGGGGCCGGGTTCACCAAACCTATCCCGGAGGGCGCGGG
CGCCCGCAC
Type: DNA
Length: 1494
OrganismName: B. megaterium strain 4B
PreSequenceString:
Sequence 9
TCTCGTCTGTCACTTAGGCGGCTAGCTCCTTTACGGTTTACTCCA
CCGACTTTCGGGTGTTACAAACTCTCGTGGTGTGACGGGCGGTGT
GTACAAGGCCCGGGAACGTATTCACCGCGGCATGCTGATCCGCGA
TTACTAGCGATTCCAGCTTCATGTAGGCGAGTTGCAGCCTACAAT
CCGAACTGAGAATGGTTTTATGGGATTGGCTTGACCTCGCGGTCT
TGCAGCCCTTTGTACCATCCATTGTAGCACGTGTGTAGCCCAGGT
CATAAGGGGCATGATGATTTGACGTCATCCCCACCTTCCTCCGGT
TTGTCACCGGCAGTCACCTTAGAGTGCCCAACTGAATGCTGGCAA
CTAAGATCAAGGGTTGCGCTCGTTGCGGGACTTAACCCAACATCT
CACGACACGAGCTGACGACAACCATGCACCACCTGTCACTCTGTC
CCCCGAAGGGGAACGCTCTATCTCTAGAGTTGTCAGAGGATGTCA
AGACCTGGTAAGGTTCTTCGCGTTGCTTCGAATTAAACCACATGC
TCCACCGCTTGTGCGGGCCCCCGTCAATTCCTTTGAGTTTCAGTC
TTGCGACCGTACTCCCCAGGCGGAGTGCTTAATGCGTTAGCTGCA
GCACTAAAGGGCGGAAACCCTCTAACACTTAGCACTCATCGTTTA
CGGCGTGGACTACCAGGGTATCTAATCCTGTTTGCTCCCCACGCT
TTCGCGCCTCAGCGTCAGTTACAGACCAAAAAGCCGCCTTCGCCA
CTGGTGTTCCTCCACATCTCTACGCATTTCACCGCTACACGTGGA
ATTCCGCTTTTCTCTTCTGCACTCAAGTTCCCCAGTTTCCAATGA
CCCTCCACGGTTGAACCGTGGGCTTTCACATCAGACTTAAGAAAC
CGCCTGCGCGCGCTTTACGCCCAATAATTCCGGATAACGCTTGCC
ACCTACGTATTACCGCGGCTGCTGGCACGTAATTAGCCCTGGCTT
TCTGGTTAGGTACCGTCAAGGTACAAGCAGTTACTCTTGTACTTG
TTCTTCCTAACAACAGAGTTTTACGACCCGAAAGCCTTCATCACT
CACGCGGCGTTGCTCCGTCAGACTTTCGTCCATTGCGGAAGATTC
CTACTGCTGCCTCCCGTAAGAATCTGGGCCGTGTCTCAATCCCAG
TGTGGCCATCACCCTCTCAGTGGGCAATGCTCCGTGCCTTGGTGA
GCGTTACTCACCACTAGCTAAGGGCCGCGGGGCCTCCTTTATTGA
AACCCAAACAACTTTTATCTTTCCTCGGAAGAGAAGACCTTCCCG
GTTAATTTCGTTTCCCAATTATCCAGTTTAAGCGAGTGGCCCCTG
TATCACCGTCGCCCCAGCTAAAAAAGTTTAAAATTGCGTCGTAAG
CGGCCGGGGGGTTTTTAGATAAGAAGTAGAAAAAAGAAAAAAGAA
GAAGGAAGTGAAGAGAAAGAGCGAAAGGAAAAGAAAAAACAGGAC
GATGAAAGACAAGAAAGAAAAGTGGTACAAAAAGGGAATTTAATA
AAAAGATAGGAAATACAAACATAAAATTCAAAATATAAAGATAAG
AGACCTAAAATAAATACAATAGATTAGATCTAATTAAATGAAATA
GATATAGATAGTATTTGGTAAAGCTGACTAATAGCGAGTAGATTG
ATATGAAACGTCTTCATTTGTTAAGCTTACATTCAGGTGTC
Type: DNA
Length: 1706
OrganismName: B. pumulis strain R2E
PreSequenceString:
Sequence 10
AGGCATGCGGCGTGCTATACATGCAGTCGAGCGGACAGAAGGGAG
CTTGCTCCCGGATGTTAGCGGCGGACGGGTGAGTAACACGTGGGT
AACCTGCCTGTAAGACTGGGATAACTCCGGGAAACCGGAGCTAAT
ACCGGATAGTTCCTTGAACCGCATGGTTCAAGGATGAAAGACGGT
TTCGGCTGTCACTTACAGATGGACCCGCGGCGCATTAGCTAGTTG
GTGGGGTAATGGCTCACCAAGGCGACGATGCGTAGCCGACCTGAG
AGGGTGATCGGCCACACTGGGACTGAGACACGGCCCAGACTCCTA
CGGGAGGCAGCAGTAGGGAATCTTCCGCAATGGACGAAAGTCTGA
CGGAGCAACGCCGCGTGAGTGATGAAGGTTTTCGGATCGTAAAGC
TCTGTTGTTAGGGAAGAACAAGTGCGAGAGTAACTGCTCGCACCT
TGACGGTACCTAACCAGAAAGCCACGGCTAACTACGTGCCAGCAG
CCGCGGTAATACGTAGGTGGCAAGCGTTGTCCGGAATTATTGGGC
GTAAAGGGCTCGCAGGCGGTTTCTTAAGTCTGATGTGAAAGCCCC
CGGCTCAACCGGGGAGGGTCATTGGAAACTGGGAAACTTGAGTGC
AGAAGAGGAGAGTGGAATTCCACGTGTAGCGGTGAAATGCGTAGA
GATGTGGAGGAACACCAGTGGCGAAGGCGACTCTCTGGTCTGTAA
CTGACGCTGAGGAGCGAAAGCGTGGGGAGCGAACAGGATTAGATA
CCCTGGTAGTCCACGCCGTAAACGATGAGTGCTAAGTGTTAGGGG
GTTTCCGCCCCTTAGTGCTGCAGCTAACGCATTAAGCACTCCGCC
TGGGGAGTACGGTCGCAAGACTGAAACTCAAAGGAATTGACGGGG
GCCCGCACAAGCGGTGGAGCATGTGGTTTAATTCGAAGCAACGCG
AAGAACCTTACCAGGTCTTGACATCCTCTGACAACCCTAGAGATA
GGGCTTTCCCTTCGGGGACAGAGTGACAGGTGGTGCATGGTTGTC
GTCAGCTCGTGTCGTGAGATGTTGGGTTAAGTCCCGCAACGAGCG
CAACCCTTGATCTTAGTTGCCAGCATTCATTTGGGCACTCTAAGG
TGACTGCCGGTGACAAACCGGAAGAAAGGTGGGGATGACGTCCAA
TCATCATGCCCCTTATGACCTGGGCTACCACGTGCTACAATGGAC
AGAACAAAGGGTTGCAGAACCGCAAGGTTTACCCATTCCCAAAAT
CTTTTTCCATTTCGGATCCAATTCTGCAATCCACCGCCTGAAACT
GGAATCCCTTAAATCCCGAAACACCAGCCGCCGGTAAAAATTTCC
CGGCCTTTTAACCCCCCGGTCACCCCGAGATTTTGACCCCCCAAA
CCTGGGTGACCTTTGGACCCGCCCCAGTGGCCATAGGTTTAAGTT
TTCCATTCAGTCAGGCCAAAACGGGGGGCCGGCCGGGAAAAAAAG
GATATAAGAAACACCCACTTGGGGCGCGGGTTTGTTTGTGTTGGG
GTATGAGTTTCTGGAGTTTATATCTTTGCTGCGGACGCTTTCCGC
GCCCT
Type: DNA
Length: 1580
OrganismName: Bacillus aryabhattai strain R2A
PreSequenceString:
Sequence 11
GAGAGGCGAAAACCGGTGACGCAAAATATTAAAACGCCAAAAAAG
AAAAAGCCGGTTAAACCGGCCCAACCCTTAAATTTGGCGAAAAAA
AATTTATTTTTTTTTCTTTTGTCTTTTTTTTCTTTCCAACAATTT
TTTTTTTTTGTTATGTTTTTTTTTTCGCTTGGGGTTTGTTCACCG
GCGGAAGGGGGGGCCCAGGCCTTTTACGAAAATAAAAAAAAAAAT
TTAAAAGTTTTTTTTTTAAGTTGGAGGCGGGGAGGGGGAATAAAA
CGGTGGGCAACCCCGCCTTTAAAGGGGGGATAAATTTGGGAAAAC
GGAAATTTAAACCGGGAAAGTATTTTTCCCTTTAGGGGGAATTAT
AAAAAGATGTTTTCGGTTTACAATTAAAAGGGGGCCCGCGGGGCC
TTAAACTAGTTGGGGAGGAAACGGTTTCACCAAGGGCAAGGGTGG
AATGGCCGACCTTGAGGGGGTATTGGCCCACACTGGGATTGAGAC
CCGGCCCCAGATTCTTACGGGGGGCAGCAGTAGGGAATCTTCCGC
AATGGACGAAAGTCTGACGGAGCAACGCCGCTTGAGTGATGAAGG
CTTTCGGGTCGTAAAACTCTGTTGTTAGGGAAGAACAAGTACGAG
AGTAACTGCTTGTACCTTGACGGTACCTAACCAGAAAGCCACGGC
TAACTACGTGCCAGCAGCCGCGGTAATACGTAGGTGGCAAGCGTT
ATCCGGAATTATTGGGCGTAAAGCGCGCGCAGGCGGTTTCTTAAG
TCTGATGTGAAAGCCCACGGCTCAACCGTGGAGGGTCATTGGAAA
CTGGGGAACTTGAGTGCAGAAGAGAAAAGCGGAATTCCACGTGTA
GCGGTGAAATGCGTAGAGATGTGGAGGAACACCAGTGGCGAAGGC
GGCTTTTTGGTCTGTAACTGACGCTGAGGCGCGAAAGCGTGGGGA
GCAAACAGGATTAGATACCCTGGTAGTCCACGCCGTAAACGATGA
GTGCTAAGTGTTAGAGGGTTTCCGCCCTTTAGTGCTGCAGCTAAC
GCATTAAGCACTCCGCCTGGGGAGTACGGTCGCAAGACTGAAACT
CAAAGGAATTGACGGGGGCCCGCACAAGCGGTGGAGCATGTGGTT
TAATTCGAAGCAACGCGAAGAACCTTACCAGGTCTTGACATCCTC
TGACAACTCTAGAGATAGAGCGTTCCCCTTCGGGGGACAGAGTGA
CAGGTGGTGCATGGTTGTCGTCAGCTCGTGTCGTGAGATGTTGGG
TTAAGTCCCGCAACGAGCGCAACCCTTGATCTTAGTTGCCAGCAT
TTAGTTGGGCACTCTAAGGTGACTGCCGGTGACAAACCGGAGGAA
GGTGGGGATGACGTCAAATCATCATGCCCCTTATGACCTGGGCTA
CACACGTGCTACAATGGATGGTACAAAGGGCTGCAAGACCGCGAG
GTCAAGCCAATCCCATAAAACCATTCTCAGTTCGGATTGTAGGCT
GCAACTCGCCTACATGAAGCTGGAATCGCTAGTAATCGCGGATCA
GCATGCCGCGGTGAATACGTTCCCGGGCCTTGTACACACCGCCCG
TCACACCACGAGAGTTTGTAACACCCGAAGTCGGTGGAGTAACCG
TAAGGAGCTAGCCGCCTAAGTGACAGATGT
Type: DNA
Length: 1650
OrganismName: Bacillus sp. strain R2D
PreSequenceString:
Sequence 12
TCTCGACCTGAGGTATGCCATTGGACTGACTGTCCGAGTCCGAGG
ACGATTGAGAATTTCCAAAGGGAAGTGCAGGGACAACCGCGGGGA
GATGAAAAGCTCTCGGTGCTTACAGTCTTGTTAGGAGGAGACACG
ATCGTAGTAATAGTGGCTGCACTGAGAGGGCTCTCACGAAGAAAG
ACCGGGCATAATGTGTCAGCAGCAGCTGTATAATATAGGTGTGGA
GCAGCTGTCTCGCAGATAATAGTGCGCAAAAGGGGCGCGGAGGGT
CTTAATAGTGTGTGTAAGACGCCCCGCGTCCTCAACCGTGGAGGG
TCATTGGAAACTGGGAGACTTGAGTGCAGAAGAGGAAAGTGGAAT
TCCATGTGTAGCGGTGAAATGCGTAGAGATATGGAGGAACACCAG
TGGCGAAGGCGACTTTCTGGTCTGTAACTGACACTGAGGCGCGAA
AGCGTGGGGAGCAAACAGGATTAGATACCCTGGTAGTCCACGCCG
TAAACGATGAGTGCTAAGTGTTAGAGGGTTTCCGCCCTTTAGTGC
TGAAGTTAACGCATTAAGCACTCCGCCTGGGGAGTACGGCCGCAA
GGCTGAAACTCAAAGGAATTGACGGGGGCCCGCACAAGCGGTGGA
GCATGTGGTTTAATTCGAAGCAACGCGAAGAACCTTACCAGGTCT
TGACATCCTCTGAAAACCCTAGAGATAGGGCTTCTCCTTCGGGAG
CAGAGTGACAGGTGGTGCATGGTTGTCGTCAGCTCGTGTCGTGAG
ATGTTGGGTTAAGTCCCGCAACGAGCGCAACCCTTGATCTTAGTT
GCCATCATTAAGTTGGGCACTCTAAGGTGACTGCCGGTGACAAAC
CGGAGGAAGGTGGGGATGACGTCAAATCATCATGCCCCTTATGAC
CTGGGCTACACACGTGCTACAATGGACGGTACAAAGAGCTGCAAG
ACCGCGAGGTGGAGCTAATCTCATAAAACCGTTCTCAGTTCGGAT
TGTAGGCTGCAACTCGCCTACATGAAGCTGGAATCGCTAGTAATC
GCGGATCAGCATGCCGCGGTGAATACGTTCCCGGGCCTTGTACAC
ACCGCCCGTCACACCACGAGAGTTTGTAACACCCGAAGTCGGTGG
GGTAACCTTTATGAGCCAGCCGCCTAAGTGACAGAGTGTGC
Type: DNA
Length: 1166
OrganismName: B. megaterium strain 4C
PreSequenceString:
Sequence 13
TCAGTCGTTACCTTAGGCGGCTAGCTCCTTTCCGGTTTCCTCCAC
CGACTTACGGGTGTTACAAACTCTCGTGGTGTGACGGGCGGTGTG
TACAAGGCCCGGGAACGTATTCACCGCGGCATGCTGATCCGCGAT
TACTAGCGATTCCAGCTTCATGTAGGCGAGTTGCAGCCTACAATC
CGAACTGAGAATGGTTTTATGGGATTGGCTTGACCTCGCGGTCTT
GCAGCCCTTTGTACCATCCATTGTAGCACGTGTGTAGCCCAGGTC
ATAAGGGGCATGATGATTTGACGTCATCCCCACCTTCCTCCGGTT
TGTCACCGGCAGTCACCTTAGAGTGCCCAACTAAATGCTGGCAAC
TAAGATCAAGGGTTGCGCTCGTTGCGGGACTTAACCCAACATCTC
ACGACACGAGCTGACGACAACCATGCACCACCTGTCACTCTGTCC
CCCGAAGGGGAACGCTCTATCTCTAGAGTTGTCAGAGGATGTCAA
GACCTGGTAAGGTTCTTCGCGTTGCTTCGAATTAAACCACATGCT
CCACCGCTTGTGCGGGCCCCCGTCAATTCCTTTGAGTTTCAGTCT
TGCGACCGTACTCCCCAGGCGGAGTGCTTAATGCGTTAGCTGCAG
CACTAAAGGGCGGAAACCCTCTAACACTTAGCACTCATCGTTTAC
GGCGTGGACTACCAGGGTATCTAATCCTGTTTGCTCCCCACGCTT
TCGCGCCTCAGCGTCAGTTACAGACCAAAAAGCCGCCTTCGCCAC
TGGTGTTCCTCCACATCTCTACGCATTTCACCGCTACACGTGGAA
TTCCGCTTTTCTCTTCTGCACTCAAGTTCCCCAGTTTCCAATGAC
CCTCCACGGTTGAACCGTGGGCTTTCACATCAGACTTAAGAAACC
GCCTGCGCGCGCTTTACGCCCAATAATTCCGGATAACGCTTGCCA
CCTACGTATTACCGCGGCTGCTGGCACGTAGTTAGCCGTGGCTTT
CTGGTTAGGTACCGTCAAGGTACGAGCAGTTACTCTCGTACTTGT
TCTTCCTAACAACAGAGTTTTACGACCCGAAAGCCTTCATCACTC
AAGCGGCGTTGCTCCGTCAGACTTTCGTCCATTGCGGAAGATCCC
TACTGCTGCCTCCCGTAGGAATCTGGGCCGTGTCTCATTCCCCAG
TTGGGCGATAACCTTCTCAGGCGGCTATGCATCGTTGCCTTGGTG
AGCCGTTATTCACCAACAACTTATGGACGCCGGCCCCTCTTTAAT
GTTAACCGAAACCTCTTTAATCATCTCCCTGAAGAGAAGAAACAT
TCCCGTTTAATCTCCGTTTCCCAAATTATCCACTCTACAGCAGGT
GGCCACGTGTTTCCCCTTCCCGCTCAGCTAAAAAAATTTAAATTT
CCATCTTAACGGGGGGGGGGTCCGCGCCCCCTTAATTCAAAAAAA
AAAAAAAGAGGGAAGCCTTACATAATATACATATAATCTATTTGA
AGACAAGGACAAAACGCTAGAATATATAAAGAACGAAAGCCCTTA
GCGTCCCTGTCCTGGCCGGGTTTGCGACCGGCCTATTACCTGGGC
CCCCGTGACGTGGTCGTAAACCGAAGCTGAGCCGCTTGGCTTGAA
CCGGGTATTGGCCCTAATTATATAGTGGTAGATGGGTTACTATGG
ACATTGTGAATGGCCAGTCACGTGC
Type: DNA
Length: 1690
OrganismName: Enterobacter asburiae strain 6A
PreSequenceString:
Sequence 14
TTATTAGTTCATTATTTTTCTGAATAAGGGTTAATTTTATTAGTT
GTTTTTTTTTTCGAAGGGGGGGGGCACAAGAAGGGAAGGCTTCGG
GTTGTAAAGTACTTTCAGCGGGAGAAGTGATGAGATATACCTCAG
CGATGACGTTACCCGCAGAAGAAGCACCGGCTAACTCCGTGCCAG
CAGCCGCGGTAATACGGAGGGTGCAAGCGTTAATCGGAATTACTG
GGCGTAAAGCGCACGCAGGCGGTCTGTCAAGTCGGATGTGAAATC
CCCGGGCTCAACCTGGGAACTGCATTCGAAACTGGCAGGCTAGAG
TCTTGTAGAGGGGGGTAGAATTCCAGGTGTAGCGGTGAAATGCGT
AGAGATCTGGAGGAATACCGGTGGCGAAGGCGGCCCCCTGGACAA
AGACTGACGCTCAGGTGCGAAAGCGTGGGGAGCAAACAGGATTAG
ATACCCTGGTAGTCCACGCCGTAAACGATGTCGACTTGGAGGTTG
TTCCCTTGAGGAGTGGCTTCCGGAGCTAACGCGTTAAGTCGACCG
CCTGGGGAGTACGGCCGCAAGGTTAAAACTCAAATGAATTGACGG
GGGCCCGCACAAGCGGTGGAGCATGTGGTTTAATTCGATGCAACG
CGAAGAACCTTACCTACTCTTGACATCCAGAGAACTTTCCAGAGA
TGGATTGGTGCCTTCGGGAACTCTGAGACAGGTGCTGCATGGCTG
TCGTCAGCTCGTGTTGTGAAATGTTGGGTTAAGTCCCGCAACGAG
CGCAACCCTTATCCTTTGTTGCCAGCGGTTCGGCCGGGAACTCAA
AGGAGACTGCCAGTGATAAACTGGAGGAAGGTGGGGATGACGTCA
AGTCATCATGGCCCTTACGAGTAGGGCTACACACGTGCTACAATG
GCGCATACAAAGAGAAGCGACCTCGCGAGAGCAAGCGGACCTCAT
AAAGTGCGTCGTAGTCCGGATTGGAGTCTGCAACTCGACTCCATG
AAGTCGGAATCGCTAGTAATCGTAGATCAGAATGCTACGGTGAAT
ACGTTCCCGGGCCTTGTACACACCGCCCGTCACACCATGGGAGTG
GGTTGCAAAAGAAGTAGGTAGCTTAACCTTCGGGAGGGCGCTACC
A
Type: DNA
Length: 1126
OrganismName: Massilia sp. strain RIC
PreSequenceString:
Sequence 15
AGGCATGGCGGCATGCTATCCATGCAGTCGTACCAGTGAGATAGC
GCGAAGCTTGCTCCGCTGACGAGTGGCGAACGGGTGAGTAATATA
TCGGAACGTACCCAAGAGTGGGGGATAACGTAGCGAAAGTTACGC
TAATACCGCATACGATCTAAGGATGAAAGCAGGGGACCTTCGGGC
CTTGTGCTCCTGGAGCGGCCGATATCTGATTAGCTAGTTGGTGAG
GTAAAGGCTCACCAAGGCGACGATCAGTAGCTGGTCTGAGAGGAC
GACCAGCCACACTGGAACTGAGACACGGTCCAGACTCCTACGGGA
GGCAGCAGTGGGGAATTTTGGACAATGGGCGCAAGCCTGATCCAG
CAATGCCGCGTGAGTGAAGAAGGCCTTCGGGTTGTAAAGCTCTTT
TGTCAGGGAAGAAACGGTCGAGGCTAATATCCTTTGCTAATGACG
GTACCTGAAGAATAAGCACCGGCTAACTACGTGCCAGCAGCCGCG
GTAATACGTAGGGTGCAAGCGTTAATCGGAATTACTGGGCGTAAA
GCGTGCGCAGGCGGTTTTGTAAGTCTGTCGTGAAATCCCCGGGCT
CAACCTGGGAATTGCGATGGAGACTGCAAGGCTAGAATCTGGCAG
AGGGGGGTAGAATTCCACGTGTAGCAGTGAAATGCGTAGAGATGT
GGAGGAACACCGATGGCGAAGGCAGCCCCCTGGGTCAAGATTGAC
GCTCATGCACGAAAGCGTGGGGAGCAAACAGGATTAGATACCCTG
GTAGTCCACGCCCTAAACGATGTCTACTAGTTGTCGGGTTTTAAT
TAACTTGGTAACGCAGCTAACGCGTGAAGTAGACCGCCTGGGGAG
TACGGTCGCAAGATTAAAACTCAAAGGAATTGACGGGGACCCGCA
CAAGCGGTGGATGATGTTGGATTAATTCGATGCAACGCGAAAAAC
CTTACCTACCCTTGACATGTCAGGAATGCTGAAGAGATTTAGCAG
TGCCCGAAAGGGAACCTGAACACAGGTGCTGCATGGCTGTCGTCA
GCTCGTGTCGTGAGATGTTGGGTTAAGTCCCGCAACGAGCGCAAC
CCTTGTCATTAGTTGCTACGCAAGAGCACTCTAATGAGACTGCCG
GTGACAAACCGGAGGAAAGGTGGGGATGACGTCAAGTCCTCATGG
CCCTTATGGGTAGGGCTTCCCACGTCATACAATGGTACATACAAA
GGGCCCCCACCCCCCGAGGGGGGCTAATCCCCAAAAATGTTTCTT
ATTCCGGATCCGATTTGGCAACTCACACCCTGAAATTGGAACCCC
CTTTAATCCCGGATAAACATGCCCCCCGTGATAAAATTCCCCGGC
TTTTTACAACCGCCGGTCAACCATGGGAGACGGTTTTTCCAAAAA
AGGGGGTTTACCCCCAGAGGGGCCTCCCCCGGGATTGTTCGGGGG
GGGGTTATAACAAAGTTAGGGAAAAGGGGGGGGGGGCCCCCTTAA
AAAAAAAAAAAAAAAAATAAAAAAAAAAATATTTTTTATCTCTTA
TTTGGCCCATTATTTCTATTTT
Type: DNA
Length: 1552
OrganismName: Ralstonia pickettii strain R3C
PreSequenceString:
Sequence 16
CGGCATGGCGGGCATGCCTTACCATGCAGTCGAACGGCAGCATGA
TCTAGCTTGCTAGATTGATGGCGAGTGGCGAACGGGTGAGTAATA
CATCGGAACGTGCCCTGTAGTGGGGGATAACTAGTCGAAAGATTA
GCTAATACCGCATACGACCTGAGGGTGAAAGTGGGGGACCGCAAG
GCCTCATGCTATAGGAGCGGCCGATGTCTGATTAGCTAGTTGGTG
AGGTAAAGGCTCACCAAGGCGACGATCAGTAGCTGGTCTGAGAGG
ACGATCAGCCACACTGGGACTGAGACACGGCCCAGACTCCTACGG
GAGGCAGCAGTGGGGAATTTTGGACAATGGGCGAAAGCCTGATCC
AGCAATGCCGCGTGTGTGAAGAAGGCCTTCGGGTTGTAAAGCACT
TTTGTCCGGAAAGAAATGGCTCTGGTTAATACCTGGGGTCGATGA
CGGTACCGGAAGAATAAGGACCGGCTAACTACGTGCCAGCAGCCG
CGGTAATACGTAGGGTCCAAGCGTTAATCGGAATTACTGGGCGTA
AAGCGTGCGCAGGCGGTTGTGCAAGACCGATGTGAAATCCCCGAG
CTTAACTTGGGAATTGCATTGGTGACTGCACGGCTAGAGTGTGTC
AGAGGGGGGTAGAATTCCACGTGTAGCAGTGAAATGCGTAGAGAT
GTGGAGGAATACCGATGGCGAAGGCAGCCCCCTGGGATAACACTG
ACGCTCATGCACGAAAGCGTGGGGAGCAAACAGGATTAGATACCC
TGGTAGTCCACGCCCTAAACGATGTCAACTAGTTGTTGGGGATTC
ATTTCCTTAGTAACGTAGCTAACGCGTGAAGTTGACCGCCTGGGG
GAGTACGGTCGCAAGATTAAAACTCAAAGGAATTGACGGGGACCC
GCACAAGCGGTGGATGAATGTGGATTAATTCGATGCAACGCGAAA
AACCTTACCTACCCTTGACATGCCACTAACGAAGCAGAGATGCAT
TAGGTGCTCGAAAGAGAAAGTGGACACAGGTGCTGCATGGCTGTC
GTCAGCTCGTGTCCTGAGAAGTTTGGGTTAAGTCCCGCAACGAGC
GCAACCCTTGTCTCTAGTTGCTACGAAAGGGCACTCTTAAAGAGA
CTGCCCGTGACAAACCCGAAGAAAGGTGGGGAATAAAATCCAATC
CTCCCGGCCCCTTAGGGGTGGGGCTTCCACCTCCTACAATGGTGG
CAACAAAAGGGTGTCCAACCCCCCCGGTGGGAGCTATTCCCAAAA
AAGGATCCTAATTTCCGGAACTAATTCTGCACTCCCACACCTTGA
AACTGGAAACTCTTTAATCCCGAATAACAAGCCCCGCGGAAAAAA
TTTCCCCGGTTTGTTATACCACCGCTCACCCCCGGGGATGGGGTG
TTCCAAATATTTATCTACCCCCAAGGCGTACCAGTTAGTTTGGGT
GGTTTAGTAAGTTTTCGCTTCCAGGGAACCAAACCGAGGGGGGGG
GGCCGGGGGGGGGGGGGGGGGGGGTAAAAAAAAAGATTGAATAAA
TGGAGGTGATAGAGTTCGGGTGGTTTTTGGTTTTGGGTGTTGGCG
TGGAATGAGGCGAAGGTTGGAAGTGGGGCTGAAATGGGAAATGGG
AAGTGGGGTAAGGGTTGTAGTTGTTTAATTGTTAAATACCCTTTG
AATTGAGAA
Type: DNA
Length: 1674
OrganismName: Enterobacter sp. Strain R4A
PreSequenceString:
Sequence 17
CCCCCTCAATCAAAGTGGGTAGCGCCCTCCCGAAGGTTAAGCTAC
CTACTTCTTTTGCAACCCACTCCCATGGTGTGACGGGCGGTGTGT
ACAAGGCCCGGGAACGTATTCACCGTAGCATTCTGATCTACGATT
ACTAGCGATTCCGACTTCATGGAGTCGAGTTGCAGACTCCAATCC
GGACTACGACGCACTTTATGAGGTCCGCTTGCTCTCGCGAGGTCG
CTTCTCTTTGTATGCGCCATTGTAGCACGTGTGTAGCCCTACTCG
TAAGGGCCATGATGACTTGACGTCATCCCCACCTTCCTCCAGTTT
ATCACTGGCAGTCTCCTTTGAGTTCCCGGCCGAACCGCTGGCAAC
AAAGGATAAGGGTTGCGCTCGTTGCGGGACTTAACCCAACATTTC
ACAACACGAGCTGACGACGCCATGCAGCACCTGTCTCAGAGTTCC
CGAAGGCACCAATCCATCTCTGGAAAGTTCTCTGGATGTCAAGAG
TAGGTAAGGTTCTTCGCGTTGCATCGAATTAAACCACATGCTCCA
CCGCTTGTGCGGGCCCCCGTCAATTCATTTGAGTTTTAACCTTGC
GGCCGTACTCCCCAGGCGGTCGACTTAACGCGTTAGCTCCGGAAG
CCACGCCTCAAGGGCACAACCTCCAAGTCGACATCGTTTACGGCG
TGGACTACCAGGGTATCTAATCCTGTTTGCTCCCCACGCTTTCGC
ACCTGAGCGTCAGTCTTTGTCCAGGGGGCCGCCTTCGCCACCGGT
ATTCCTCCTGATCTCTACGCATTTCACCGCTACACCTGGAATTCT
ACCCCCCTCTACAAGACTCTCGCCTGCCGGTTTCGAATGCAGTTC
CCACGTTGAGCCCGGGGATTTCACATCCCACTTGACAGACCGCCG
GCGTGCGCTTTACGCCCAGTAATTCCGATTAACGCTTGCACCCTC
CGTATTACCGCGGCTGCTGGCACGGAGTTAGCCGGTGCTTCTTCT
GCGGGTAACGTCAATCGACAAGGTTATTAGCCTTATCGCCTTCCT
CCCCGCTGAAAGTACTTTACAACCCGAAGGGCTTTCTTCTTACAC
CCGGGCAGGGTTGCATCAGGCTTGCCCCCATTGTGCAATATTCCC
CAATGCTGGCGTCCCGTAAGAATTTGGGACCGGGTCTAATTTCCA
GGGTGGCTGGGCATCCCCTCAAACCAGCTAGGGAACCTTCCCCAA
GGTGAACCCGTTACCGACCTACCAGCCTAATTCCAATCGGGGGCC
ATCCGAAAGGCCAAAAGGCCGAAAGGTCCCCCCCTTTTGTTCTTG
CGACCTTTTGGCGGTATTGACCCACCGGTTCCCGAAATTTACCCC
CCTCCCTACGGGGAATTTCCCAAACATTTCTCCCCCCCCCCCCCC
CCCTGGCCCCCAAGAAAAACTCGTTTTTTCAACCCTTCTTTCCTT
TGTTTAGGCCCCGGCGGACGTTTAAATTTTAACCCCAAGGCTAAA
TCTAAGAAAAAACCTCCCCCCGGGCCCCTAAAAGGGGAATATCAC
GGGTTTGCGCCATGGGTAAAGGGGACAGGGGTTAAAAAGTTTGGC
AG
Type: DNA
Length: 1577

1.-52. (canceled)

53. A method for improving a plant trait, comprising steps of:
a. obtaining a plant or a seed; and
b. inoculating said plant or said seed with at least one bacterium having at least one of sequences SEQ ID NO:1-SEQ ID NO: 17, as disclosed in the sequence listing of said application.

54. The method of claim 53, further comprising steps of germinating said seed and growing a plant from said seed.

55. A bacterial formulation comprising at least one bacterium having at least one of sequences SEQ ID NO:1-SEQ ID NO:17, as disclosed in the sequence listing of said application.

56. The bacterial formulation of claim 55, wherein said formulation comprises bacteria selected from a group consisting of Bacillus spp, Enterobacter spp, Massilia spp, Pseudomonas spp, Ralstonia spp and any combination thereof.

57. The bacterial formulation of claim 55 for use in improving a plant trait.

58. The bacterial formulation of claim 57, wherein said plant is selected from the group consisting of: a tomato plant and a seedling.

59. The bacterial formulation of claim 57, wherein said plant trait is characterized by an increase of at least about 2% compared to plant of the same species not treated with said bacterial formulation.

60. The bacterial formulation of claim 57, wherein said plant trait is selected from a group consisting of improved height, increased number of flowers, increases number of fruits, improved plant weight, increased harvest index, activation of an immune response, increased yield, enhanced resistance to pathogenic infections and any combination thereof.

61. The bacterial formulation of claim 60, wherein said enhanced resistance to pathogenic infections is characterized by a decrease in a lesion area, decrease in tissue damage, or the number of pathogenic microorganisms of at least about 2%.

62. The bacterial formulation of claim 60, wherein said pathogenic infections are caused by microorganisms, selected from the group consisting of bacteria, fungi, viruses and any combination thereof.

63. The method of claim 60, wherein said plant has not yet set fruit.

64. The method of claim 60, wherein said plant has set fruit.

65. The method of claim 60, wherein said plant is 3-6 weeks old.

66. The method of claim 60, wherein said plant is selected from a group consisting of a Solanaceous plant, a Cucurbit plant, and a Vitaceae plant.

67. The method of claim 53, wherein said plant trait is characterized by an increase of at least about 2% compared to uninoculated plant of the same species.

68. The method of claim 53, wherein said plant trait is activation of an immune response, said activation of an immune response is measured by a mean selected from a group consisting of production of ethylene, generation of reactive oxygen species, expression of genes related to the plant immune and defense response and any combination thereof.

69. The method of claim 53, wherein said plant trait is enhanced resistance to pathogenic infection caused by a microorganism, said microorganism is selected from the group consisting of bacteria, fungi, viruses, and any combination thereof.

70. The method of claim 69, wherein said pathogenic infection is selected from the group consisting of Xanthomonas campestris pv. Vesicatoria, Botrytis cinerea, and any combination thereof.

71. The method of claim 69, wherein said pathogenic infection is a bacterium selected from the group consisting of Bacillus spp, Enterobacter spp, Massilia spp, Pseudomonas spp, Ralstonia spp, and any combination thereof.

72. The method of claim 71, wherein said bacterium is selected from a group consisting of Bacillus cereus, Bacillus licheninformis, Bacillus subtilis, Bacillus megaterium, Bacillus aryabhattai, Bacillus pumulis, Enterobacter asburiae, Ralstonia pickettii, Pseudomonas aeruginosa, and any combination thereof.


SEQUENCE LISTING (17 sequences)

SEQ ID NO: 1 - DNA, 1661 bp, Bacillus sp. strain R3B
tccttgtcacttaggcggctagctccttacggttactccaccgacttcgggtgttacaaactctcgtggtgtgacgggcggtgtgtacaaggcccgggaacgtattcaccgcggcatgctgatccgcgattactagcgattccagcttcatgtaggcgagttgcagcctacaatccgaactgagaatggttttatgggattggcttgacctcgcggtcttgcagccctttgtaccatccattgtagcacgtgtgtagcccaggtcataaggggcatgatgatttgacgtcatccccaccttcctccggtttgtcaccggcagtcaccttagagtgcccaactaaatgctggcaactaagatcaagggttgcgctcgttgcgggacttaacccaacatctcacgacacgagctgacgacaaccatgcaccacctgtcactctgtcccccgaaggggaacgctctatctctagagttgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaagggcggaaaccctctaacacttagcactcatcgtttacggcgtggactaccagggtatctaatcctgtttgctccccacgctttcgcgcctcagcgtcagttacagaccaaaaagccgccttcgccactggtgttcctccacatctctacgcatttcaccgctacacgtggaattccgcttttctcttctgcactcaagttccccagtttccaatgaccctccacggttgagccgtgggctttcacatcagacttaagaaaccgcctgcgcgcgctttacgcccaataattccggataacgcttgccacctacgtattaccgcggctgctggcacgtagttagccgtggctttctggttaggtaccgtcaaggtacaagcagttactcttgtacttgttcttccctaacaacagaattttacgacccgaaagccttcatcactcaagcggcgttgctccgtcagactttcgtccattgcggaagattccctactgctgccccccctaagaatctggggccgggtctcaatccccctgtgggccaaaaccccctccaggtcgcccatgaaacctttgccttggtgaaccgttacctcccaactaacttatggccccccgcgcccccctttaatgtataacccaaaccaatttttattaactcccccgaaagggaaaaatcaattccggtttaatttcgggtttcccaaattttcccacctttaaaggagggttgccccacgtttatcacccttcccccctcattctaaaaaaaaactttaaatttttcgaattggtaaggctggccccccccgggcgggactcactcaggtaaaaaaaaaaaaattatgaaccctaaaaaaataaacccccgggccaccaaaatattaaagaacccaaaaacaaaaaaattgaaccgtttcccctggccctgtttgtgtgttgttgatgcgggccggtgtaaacgggtatttggtgacccctgggctttggtggccgctgtaatggcgtgatgtttgttttttggtgttt

SEQ ID NO: 2 - DNA, 1178 bp, Bacillus sp. strain R4B
gaatgggggggggttgctataatgcagtcgagcgatcgatggtagcttggtccttcagattatcgggggatgggtgagtgtgcgtgggggcccggcctgtaagacagggacaacttcgggaaaccggagctaataccggatattttcttttctcacatgagataaaatggagagacggtttatggtctcgggggacccgttgcccccgcggcgttggggattggttggaggccaatggcaccaaggcgacgatatggtttaggggggggggggggttggggcttacagggaccccgaaccctcccacaggcgcattcggtggagtctcccccagaaatcccgcaaaggacaagaatctccgggggcgaggccccgtgttaaataaaaaaactcgggttttgaaaaaaaaagtcggggagaaaaggcgggccattaaatggagggaaccctaagggacctcctaaaaaaagccgggcgcagaagttggcctggtcccccgtatttctctttatgtataccctttttggccggtatttggtgggttaataatgacgcccgggggttccccccccccctccccgaggaagcccccccgcttgctgcccgtgggggggcttttgggaaaaatgggaacatttccttctctcacaaaaaaatgtgatattttttcgggggggggggaaaaaaggtggaattttttcggtgagtccccgaacttcaggcacgaatcggggggagagaacaaaatagctgcgcctccactgcttttcgcaaaagatgacggcagttgaagggcgactcccggcttctcacgtctaacttcgcagacaaacactcagcccgcgggtgggctcccgcggggtcttaacccgaagtattttaagcatctcactgggagcatccgtgcgcgtgggtataacgaatagaatcccataaatctgggtaggcctggggtatgtacttaatcgtaggatagctgttcgcttgcggttttagaatggagggaggagctacggtcgtcccgtccctagcaatcaaatgttagccgtccagaaagcggacattattcaccacagaaatttctatctttcgtcagtacgtcttttcgtccctcacgtaacacgtcttcaggtccgaaagaagataccgagggaggagcgcctccactcgcattgatg

SEQ ID NO: 3 - DNA, 1500 bp, B. cereus strain 6B
cccctcccgggttttttttctttttttttgttttaaccccccccccccccccccttacctaagtggtctctcggacagaggagcacgggctttaccgattgggaaagtttaagctttttctaagattggcgggacggggaacaccgggttaccgcccaaagggtggaaattcgggaaccggggttaatccggataccttttgaaccgcatgtttgaaatgaaaggcggtttggctttcatttatggaggacccggctcgcattagctagtggggaggtaacggttcaccaaggcaacgatgggtagccgacctgagaggtgatcggccacactgggactgagacacggcccagattcttacgggaggcagcagtagggattttccgcaatggacgaaagtctgacggagcaacgccgcgtgaatgatgaaggctttcgggttgtaaaactttgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaacctttttggagccagccgcctaagtgacagtaattga

SEQ ID NO: 4 - DNA, 1400 bp, B. cereus strain R1B
gcgcatggcgggtgctatactgcaagtcgagcgaatggattaagagcttgctcttatgaagttagcggcggacgggtgagtaacacgtgggtaacctgcccataagactgggataactccgggaaaccggggctaataccggataacattttgaaccgcatggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcacgcgaagaccttaccaggtcttgactcctctgaaaccctagagataggcttctcttcggaacaaatgacagtggtgctgttgcctcactcgggccggaatgtgggtaagtccgcacgaggcaccttgactttttgccgcattatttgggacttcagggacgccgtgaaaacgggggaagggggggtactccacttttagcccttaaacggggtaaaagggttaaaagagggaaaaaactccaaaccccggttgaaaattcttaaaccttttttttgaatagggccccccccccccaaggggaaccttaaaacgaaaaaaaccccggaaattccccccttcccccccgccccccaaattaactccaaccttggaccttcccccccaggattaggttacatttaccgaaacccctacgattgaagaattgatgatagaaaaattaagtttgggtggttt

SEQ ID NO: 5 - DNA, 1408 bp, B. licheniformis strain R3D
cccctcataattaacgcagaccaccaccaagaataatacccttctcctcccttttttgattttttttttttaatggccggcccgggcactacaagggaaaggtttccgagggggggggaaaagggtacccctaaagggaatcggaacgggttaccgttttttaccctgttaaaaaagggtttgttcattaggggccgggccttattgtggggaacgctcccaggcacgtggtgccactgggggtatgcccccgggatgaaccgcccaattctagggagcgcgtaggatttcgcaaggagaagtcgacgagaacgcgctgatgagaagttgacgagcacgccgcgtgagtgatgaagttttcgatcgtaaactctgtgtagggagacagtaccgtcgatagggcggcacctgacggtacctaaccagaaagccacggctaactacgtgccagcagccgcggtaatacgtaggtggcaagcgtgtccggaattattgggcgtaaagcgcgcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttggagccagccgccgaagtgcgagttga

SEQ ID NO: 6 - DNA, 1322 bp, B. licheniformis strain F4_27F
cacatctccgcctcttcatgggagtcccaaggtctgacggagcaccgcgctgggatggatgaaggttttcggttcgtaaaactctgttgttaggagaacaagtacgtcgatagggccgtaccttgacggtacctaaccagaaagcacggctaactacgtgccagcagccgcggtaatacgtaggtggcaagcgtgtccggaattattgggcgtaaagcgcgcgcaggcggtttcttaagtctgatgtgaaagccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtcctaatctctcctcaacggcttgtcgttaagtttccctgcgaaataccccgcgctcccccggaattggcagttctccctcgggctgacactccctggagcagcggttcctgtaaaatcgctcccctctaaggccccgcgttttgtgttataaccaccctcttcgccgggttctgctccgtgacggtcctaagtccttcccgcgccgcccgtgccctccgccaacgaaatactttccctcgtagcggccgccccggcccaaa

SEQ ID NO: 7 - DNA, 1613 bp, B. subtilis strain R1D
tcactcctgtcacttcggcggctggctccataaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaaggaaccgcctgcgagccctttacgcccaataattccggacaacgctggccacctacgtattaccgcggctgctggcacgtagttagccgtggctttctggttaggtaccgtcaaggtaccgccctattcgaacggtacttgttcttccctaacaacagagctttacgatccgaaaaccttcatcactcaagcggcgttgctccgtcagactttcttccattgcggaaaaattccctactgctgcctccccctaagaaatctggggccgtgtctcaatcccccttgtgggccaacaccctctccagggcggccaaccacccctttcccttgtggaagccattactccaccaacaaattaaagggccgcggggcctcctttaattggtatccaaaccaccctttttttttttaaccaagggggttaaaaaaaaaacccgggtataaacccccgtttcccgggtatttccaccttttaagggggggtgaccccggtttatccccctcccccccctaaaaaggaaagattcaacttccccccaatccttaaagcttgggcccccgccgggtgcctacacggaaaaaaaaaaaaaaagacaaatataactaaaataacgaacccagaaaataagggagaaaaagtaaaataagatattaaagcccccccccggggggagaaattggaaaccctttaaagttttgttaaagggggtaatataaaaaac

SEQ ID NO: 8 - DNA, 1494 bp, B. cereus strain 4A
gtgctagcgggtcctatacatgcaagtcgagcgaatggattgagagcttgctctcaagaagttagcggcggacgggtgagtaacacgtgggtaacctgcccataagactgggataactccgggaaaccggggctaataccggataacattttgaactgcatggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaccccaagaaccttaccaggtcttgacatcctctgaaaaccctagagatagggcttctccttcgggagcaaagtgacaggtggtgcatgcttgtcttcatctcttgccctgaaatgtggggtaagtcccccaacaacgccacccttgatttacttgcaacataagttgggacccaaagggaatgccggggacacccggaagaagcggggatgaccttatactacatgctatatagtactgggctcacccgggttcaatggccggtcaacaattgccgacccaaggtgatcaatcccttaccggtcaattccgaggggggctaattccattgaattgaaacctttaaccggaaaccaccctttggaaaccttccggccgttaaccgacccggccggaatcttgaacccttaccggggggctttttgaccggccttggcctttttgggggtaagggcggaaacgaaacggaaagcccaaaaaatatacatatataatattccaataaggggccgggttcaccaaacctatcccggagggcgcgggcgcccgcac

SEQ ID NO: 9 - DNA, 1706 bp, B. megaterium strain 4B
tctcgtctgtcacttaggcggctagctcctttacggtttactccaccgactttcgggtgttacaaactctcgtggtgtgacgggcggtgtgtacaaggcccgggaacgtattcaccgcggcatgctgatccgcgattactagcgattccagcttcatgtaggcgagttgcagcctacaatccgaactgagaatggttttatgggattggcttgacctcgcggtcttgcagccctttgtaccatccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgtcccccgaaggggaacgctctatctctagagttgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaagggcggaaaccctctaacacttagcactcatcgtttacggcgtggactaccagggtatctaatcctgtttgctccccacgctttcgcgcctcagcgtcagttacagaccaaaaagccgccttcgccactggtgttcctccacatctctacgcatttcaccgctacacgtggaattccgcttttctcttctgcactcaagttccccagtttccaatgaccctccacggttgaaccgtgggctttcacatcagacttaagaaaccgcctgcgcgcgctttacgcccaataattccggataacgcttgccacctacgtattaccgcggctgctggcacgtaattagccctggctttctggttaggtaccgtcaaggtacaagcagttactcttgtacttgttcttcctaacaacagagttttacgacccgaaagccttcatcactcacgcggcgttgctccgtcagactttcgtccattgcggaagattcctactgctgcctcccgtaagaatctgggccgtgtctcaatcccagtgtggccatcaccctctcagtgggcaatgctccgtgccttggtgagcgttactcaccactagctaagggccgcggggcctcctttattgaaacccaaacaacttttatctttcctcggaagagaagaccttcccggttaatttcgtttcccaattatccagtttaagcgagtggcccctgtatcaccgtcgccccagctaaaaaagtttaaaattgcgtcgtaagcggccggggggtttttagataagaagtagaaaaaagaaaaaagaagaaggaagtgaagagaaagagcgaaaggaaaagaaaaaacaggacgatgaaagacaagaaagaaaagtggtacaaaaagggaatttaataaaaagataggaaatacaaacataaaattcaaaatataaagataagagacctaaaataaatacaatagattagatctaattaaatgaaatagatatagatagtatttggtaaagctgactaatagcgagtagattgatatgaaacgtcttcatttgttaagcttacattcaggtgtc

SEQ ID NO: 10 - DNA, 1580 bp, B. pumulis strain R2E
aggcatgcggcgtgctatacatgcagtcgagcggacagaagggagcttgctcccggatgttagcggcggacgggtgagtaacacgtgggtaacctgcctgtaagactgggataactccgggaaaccggagctaataccggatagttccttgaaccgcatggttcaaggatgaaagacggtttcggctgtcacttacagatggacccgcggcgcattagctagttggtggggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgcgagagtaactgctcgcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcattcatttgggcactctaaggtgactgccggtgacaaaccggaagaaaggtggggatgacgtccaatcatcatgccccttatgacctgggctaccacgtgctacaatggacagaacaaagggttgcagaaccgcaaggtttacccattcccaaaatctttttccatttcggatccaattctgcaatccaccgcctgaaactggaatcccttaaatcccgaaacaccagccgccggtaaaaatttcccggccttttaaccccccggtcaccccgagattttgaccccccaaacctgggtgacctttggacccgccccagtggccataggtttaagttttccattcagtcaggccaaaacggggggccggccgggaaaaaaaggatataagaaacacccacttggggcgcgggtttgtttgtgttggggtatgagtttctggagtttatatctttgctgcggacgctttccgcgccct

SEQ ID NO: 11 - DNA, 1650 bp, Bacillus aryabhattai strain R2A
gagaggcgaaaaccggtgacgcaaaatattaaaacgccaaaaaagaaaaagccggttaaaccggcccaacccttaaatttggcgaaaaaaaatttatttttttttcttttgtcttttttttctttccaacaattttttttttttgttatgttttttttttcgcttggggtttgttcaccggcggaagggggggcccaggccttttacgaaaataaaaaaaaaaatttaaaagttttttttttaagttggaggcggggagggggaataaaacggtgggcaaccccgcctttaaaggggggataaatttgggaaaacggaaatttaaaccgggaaagtatttttccctttagggggaattataaaaagatgttttcggtttacaattaaaagggggcccgcggggccttaaactagttggggaggaaacggtttcaccaagggcaagggtggaatggccgaccttgagggggtattggcccacactgggattgagacccggccccagattcttacggggggcagcagtagggaatcttccgcaatggacgaaagtctgacggagcaacgccgcttgagtgatgaaggctttcgggtcgtaaaactctgttgttagggaagaacaagtacgagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaagtgacagatgt

SEQ ID NO: 12 - DNA, 1166 bp, Bacillus sp. strain R2D
tctcgacctgaggtatgccattggactgactgtccgagtccgaggacgattgagaatttccaaagggaagtgcagggacaaccgcggggagatgaaaagctctcggtgcttacagtcttgttaggaggagacacgatcgtagtaatagtggctgcactgagagggctctcacgaagaaagaccgggcataatgtgtcagcagcagctgtataatataggtgtggagcagctgtctcgcagataatagtgcgcaaaaggggcgcggagggtcttaatagtgtgtgtaagacgccccgcgtcctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaacctttatgagccagccgcctaagtgacagagtgtgc

SEQ ID NO: 13 - DNA, 1690 bp, B. megaterium strain 4C
tcagtcgttaccttaggcggctagctcctttccggtttcctccaccgacttacgggtgttacaaactctcgtggtgtgacgggcggtgtgtacaaggcccgggaacgtattcaccgcggcatgctgatccgcgattactagcgattccagcttcatgtaggcgagttgcagcctacaatccgaactgagaatggttttatgggattggcttgacctcgcggtcttgcagccctttgtaccatccattgtagcacgtgtgtagcccaggtcataaggggcatgatgatttgacgtcatccccaccttcctccggtttgtcaccggcagtcaccttagagtgcccaactaaatgctggcaactaagatcaagggttgcgctcgttgcgggacttaacccaacatctcacgacacgagctgacgacaaccatgcaccacctgtcactctgtcccccgaaggggaacgctctatctctagagttgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaagggcggaaaccctctaacacttagcactcatcgtttacggcgtggactaccagggtatctaatcctgtttgctccccacgctttcgcgcctcagcgtcagttacagaccaaaaagccgccttcgccactggtgttcctccacatctctacgcatttcaccgctacacgtggaattccgcttttctcttctgcactcaagttccccagtttccaatgaccctccacggttgaaccgtgggctttcacatcagacttaagaaaccgcctgcgcgcgctttacgcccaataattccggataacgcttgccacctacgtattaccgcggctgctggcacgtagttagccgtggctttctggttaggtaccgtcaaggtacgagcagttactctcgtacttgttcttcctaacaacagagttttacgacccgaaagccttcatcactcaagcggcgttgctccgtcagactttcgtccattgcggaagatccctactgctgcctcccgtaggaatctgggccgtgtctcattccccagttgggcgataaccttctcaggcggctatgcatcgttgccttggtgagccgttattcaccaacaacttatggacgccggcccctctttaatgttaaccgaaacctctttaatcatctccctgaagagaagaaacattcccgtttaatctccgtttcccaaattatccactctacagcaggtggccacgtgtttccccttcccgctcagctaaaaaaatttaaatttccatcttaacggggggggggtccgcgcccccttaattcaaaaaaaaaaaaaagagggaagccttacataatatacatataatctatttgaagacaaggacaaaacgctagaatatataaagaacgaaagcccttagcgtccctgtcctggccgggtttgcgaccggcctattacctgggcccccgtgacgtggtcgtaaaccgaagctgagccgcttggcttgaaccgggtattggccctaattatatagtggtagatgggttactatggacattgtgaatggccagtcacgtgc

SEQ ID NO: 14 - DNA, 1126 bp, Enterobacter asburiae strain 6A
ttattagttcattatttttctgaataagggttaattttattagttgttttttttttcgaagggggggggcacaagaagggaaggcttcgggttgtaaagtactttcagcgggagaagtgatgagatatacctcagcgatgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcggttcggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgctacca

SEQ ID NO: 15 - DNA, 1552 bp, Massilia sp. strain R1C
aggcatggcggcatgctatccatgcagtcgtaccagtgagatagcgcgaagcttgctccgctgacgagtggcgaacgggtgagtaatatatcggaacgtacccaagagtgggggataacgtagcgaaagttacgctaataccgcatacgatctaaggatgaaagcaggggaccttcgggccttgtgctcctggagcggccgatatctgattagctagttggtgaggtaaaggctcaccaaggcgacgatcagtagctggtctgagaggacgaccagccacactggaactgagacacggtccagactcctacgggaggcagcagtggggaattttggacaatgggcgcaagcctgatccagcaatgccgcgtgagtgaagaaggccttcgggttgtaaagctcttttgtcagggaagaaacggtcgaggctaatatcctttgctaatgacggtacctgaagaataagcaccggctaactacgtgccagcagccgcggtaatacgtagggtgcaagcgttaatcggaattactgggcgtaaagcgtgcgcaggcggttttgtaagtctgtcgtgaaatccccgggctcaacctgggaattgcgatggagactgcaaggctagaatctggcagaggggggtagaattccacgtgtagcagtgaaatgcgtagagatgtggaggaacaccgatggcgaaggcagccccctgggtcaagattgacgctcatgcacgaaagcgtggggagcaaacaggattagataccctggtagtccacgccctaaacgatgtctactagttgtcgggttttaattaacttggtaacgcagctaacgcgtgaagtagaccgcctggggagtacggtcgcaagattaaaactcaaaggaattgacggggacccgcacaagcggtggatgatgttggattaattcgatgcaacgcgaaaaaccttacctacccttgacatgtcaggaatgctgaagagatttagcagtgcccgaaagggaacctgaacacaggtgctgcatggctgtcgtcagctcgtgtcgtgagatgttgggttaagtcccgcaacgagcgcaacccttgtcattagttgctacgcaagagcactctaatgagactgccggtgacaaaccggaggaaaggtggggatgacgtcaagtcctcatggcccttatgggtagggcttcccacgtcatacaatggtacatacaaagggcccccaccccccgaggggggctaatccccaaaaatgtttcttattccggatccgatttggcaactcacaccctgaaattggaaccccctttaatcccggataaacatgccccccgtgataaaattccccggctttttacaaccgccggtcaaccatgggagacggtttttccaaaaaagggggtttacccccagaggggcctcccccgggattgttcgggggggggttataacaaagttagggaaaagggggggggggcccccttaaaaaaaaaaaaaaaaaaataaaaaaaaaaatattttttatctcttatttggcccattatttctatttt

SEQ ID NO: 16 - DNA, 1674 bp, Ralstonia pickettii strain R3C
cggcatggcgggcatgccttaccatgcagtcgaacggcagcatgatctagcttgctagattgatggcgagtggcgaacgggtgagtaatacatcggaacgtgccctgtagtgggggataactagtcgaaagattagctaataccgcatacgacctgagggtgaaagtgggggaccgcaaggcctcatgctataggagcggccgatgtctgattagctagttggtgaggtaaaggctcaccaaggcgacgatcagtagctggtctgagaggacgatcagccacactgggactgagacacggcccagactcctacgggaggcagcagtggggaattttggacaatgggcgaaagcctgatccagcaatgccgcgtgtgtgaagaaggccttcgggttgtaaagcacttttgtccggaaagaaatggctctggttaatacctggggtcgatgacggtaccggaagaataaggaccggctaactacgtgccagcagccgcggtaatacgtagggtccaagcgttaatcggaattactgggcgtaaagcgtgcgcaggcggttgtgcaagaccgatgtgaaatccccgagcttaacttgggaattgcattggtgactgcacggctagagtgtgtcagaggggggtagaattccacgtgtagcagtgaaatgcgtagagatgtggaggaataccgatggcgaaggcagccccctgggataacactgacgctcatgcacgaaagcgtggggagcaaacaggattagataccctggtagtccacgccctaaacgatgtcaactagttgttggggattcatttccttagtaacgtagctaacgcgtgaagttgaccgcctgggggagtacggtcgcaagattaaaactcaaaggaattgacggggacccgcacaagcggtggatgaatgtggattaattcgatgcaacgcgaaaaaccttacctacccttgacatgccactaacgaagcagagatgcattaggtgctcgaaagagaaagtggacacaggtgctgcatggctgtcgtcagctcgtgtcctgagaagtttgggttaagtcccgcaacgagcgcaacccttgtctctagttgctacgaaagggcactcttaaagagactgcccgtgacaaacccgaagaaaggtggggaataaaatccaatcctcccggccccttaggggtggggcttccacctcctacaatggtggcaacaaaagggtgtccaacccccccggtgggagctattcccaaaaaaggatcctaatttccggaactaattctgcactcccacaccttgaaactggaaactctttaatcccgaataacaagccccgcggaaaaaatttccccggtttgttataccaccgctcacccccggggatggggtgttccaaatatttatctacccccaaggcgtaccagttagtttgggtggtttagtaagttttcgcttccagggaaccaaaccgaggggggggggccggggggggggggggggggggtaaaaaaaaagattgaataaatggaggtgatagagttcgggtggtttttggttttgggtgttggcgtggaatgaggcgaaggttggaagtggggctgaaatgggaaatgggaagtggggtaagggttgtagttgtttaattgttaaataccctttgaattgagaa

SEQ ID NO: 17 - DNA, 1577 bp, Enterobacter sp. Strain R4A
ccccctcaatcaaagtgggtagcgccctcccgaaggttaagctacctacttcttttgcaacccactcccatggtgtgacgggcggtgtgtacaaggcccgggaacgtattcaccgtagcattctgatctacgattactagcgattccgacttcatggagtcgagttgcagactccaatccggactacgacgcactttatgaggtccgcttgctctcgcgaggtcgcttctctttgtatgcgccattgtagcacgtgtgtagccctactcgtaagggccatgatgacttgacgtcatccccaccttcctccagtttatcactggcagtctcctttgagttcccggccgaaccgctggcaacaaaggataagggttgcgctcgttgcgggacttaacccaacatttcacaacacgagctgacgacgccatgcagcacctgtctcagagttcccgaaggcaccaatccatctctggaaagttctctggatgtcaagagtaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccacgcctcaagggcacaacctccaagtcgacatcgtttacggcgtggactaccagggtatctaatcctgtttgctccccacgctttcgcacctgagcgtcagtctttgtccagggggccgccttcgccaccggtattcctcctgatctctacgcatttcaccgctacacctggaattctacccccctctacaagactctcgcctgccggtttcgaatgcagttcccacgttgagcccggggatttcacatcccacttgacagaccgccggcgtgcgctttacgcccagtaattccgattaacgcttgcaccctccgtattaccgcggctgctggcacggagttagccggtgcttcttctgcgggtaacgtcaatcgacaaggttattagccttatcgccttcctccccgctgaaagtactttacaacccgaagggctttcttcttacacccgggcagggttgcatcaggcttgcccccattgtgcaatattccccaatgctggcgtcccgtaagaatttgggaccgggtctaatttccagggtggctgggcatcccctcaaaccagctagggaaccttccccaaggtgaacccgttaccgacctaccagcctaattccaatcgggggccatccgaaaggccaaaaggccgaaaggtcccccccttttgttcttgcgaccttttggcggtattgacccaccggttcccgaaatttacccccctccctacggggaatttcccaaacatttctccccccccccccccccctggcccccaagaaaaactcgttttttcaacccttctttcctttgtttaggccccggcggacgtttaaattttaaccccaaggctaaatctaagaaaaaacctccccccgggcccctaaaaggggaatatcacgggtttgcgccatgggtaaaggggacaggggttaaaaagtttggcag